(19) United States
(12) Patent Application Publication
(10) Pub. No.: US 2009/0137544 A1
(43) Pub. Date: May 28, 2009

(54) Formation and rejuvenation of organs and alcohol damaged organ regeneration through stem cell nutrients

(76) Inventor: Yin-Xiong Li, Durham, NC (US)

Correspondence Address: CHARLES J. PRESCOTT, P.A., SUITE 115, 2033 WOOD STREET, SARASOTA, FL, 34237, US

(21) Appl. No.: 12/001,142

(22) Filed: Dec. 10, 2007

Related U.S. Application Data
(60) Provisional application No. 60/873,826, filed on Dec. 8, 2006.
(60) Provisional application No. 60/901,945, filed on Feb. 14, 2007.
(60) Provisional application No. 60/923,927, filed on Apr. 17, 2007.

(51) Int. Cl. A61K 31/575; C12Q 1/60; A01K 67/027

(52) U.S. Cl. 514/182; 435/11; 800/3

(57) ABSTRACT

Mechanisms nourish stem cells for organ regeneration and prevent alcohol related diseases such as Fetal Alcohol Syndrome (FAS) and Liver Sclerosis. These stem cell nutrients have been found to positively affect the skin, liver, brain neurons, pancreas, and the GI tract. Cholesterol supplementation prevents fetal alcohol spectrum defects (FASD) in alcohol-exposed zebra fish embryos. Using the zebra fish model, alcohol was found to interfere with embryonic development by disrupting cholesterol-dependent activation of a critical signaling molecule, sonic hedgehog (Shh). Cholesterol supplementation of the alcohol-exposed embryos restored the functionality of the molecular pathway and prevented development of FASD-like defects. Novel biomarkers were identified for diagnosing alcohol related diseases by lipid chemical analysis and Raman Spectroscope.

### CROSS-REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

1. Field of the Invention

This invention relates generally to a breakthrough in formation and rejuvenation of organs through stem cell nutrients and alcohol damaged organ regeneration. New mechanisms have been discovered which nourish stem cells for organ regeneration and prevent alcohol related diseases such as Fetal Alcohol Syndrome (FAS) and Liver Sclerosis. These stem cell nutrients have been found to positively affect the skin, liver, brain neurons, pancreas, and the GI tract.

2. Description of Related Art

Consumption of alcohol by pregnant women can cause fetal alcohol spectrum defects (FASD), a congenital disease, which is characterized by an array of developmental defects that include neurological, craniofacial, cardiac, and limb malformations, as well as generalized growth retardation. FASD remains a significant clinical challenge and an important social problem. Although there has been great progress in delineating the mechanisms contributing to alcohol-induced birth defects, gaps in our knowledge still remain; for instance, why does alcohol preferentially induce a spectrum of defects in specific organs and why is the spectrum of defects reproducible and predictable.

Alcohol related birth defects leave around 100 babies every day in United States alone with little chance of having more than average IQ, and many with some malformed organs. The cost to the US for the care of these children is staggering at an estimated annual cost of $10 billion to the health care system. Fetal alcohol syndrome is a term used to describe an array of developmental defects affecting the nervous and cardiovascular systems. The syndrome also can lead to growth retardation, facial abnormalities and lowered mental functioning.

The keys to fetal alcohol syndrome's severity are the amount of alcohol consumed, the duration of the consumption and the timing of the pregnancy. For example, alcohol consumed by a mother with a one-month-old fetus could alter the development of the brain; at four to eight weeks, facial structures, heart and eyesight could be affected. Two to three months into fetal development, alcohol consumption could lead to the growth of extra digits. The amount of alcohol consumed is important as well. Even the equivalent of one 12-ounce beer, consumed at the wrong time, could disrupt the signaling pathway and lead to a defect. Increased amounts of alcohol exposure by the fetus lead to increased severity of the syndrome.

### BRIEF SUMMARY OF THE INVENTION

### Fetal Alcohol Syndrome

Applicant has found that cholesterol supplementation prevents fetal alcohol spectrum defects in alcohol-exposed zebra fish embryos. Using the zebra fish model, applicant has found alcohol interferes with embryonic development by disrupting cholesterol-dependent activation of a critical signaling molecule, called the sonic hedgehog (Shh). Cholesterol supplementation of the alcohol-exposed embryos restored the functionality of the molecular pathway and prevented development of such defects. Alcohol related-like defects in zebra fish resulted from minimal fetal alcohol exposure, equivalent to a 120-pound woman drinking one 12-ounce bottle of beer. The findings suggest even small amounts of alcohol might be unsafe for a pregnant woman and also indicate cholesterol supplementation may be a potential means of preventing fetal alcohol defects.

Small amounts of alcohol can interfere with the growth of a fetus, but added cholesterol may help prevent a wide array of neurological and physical defects from alcohol exposure. Cholesterol is so important to fetal development that pregnant women who do not have physiological high enough cholesterol levels are at increased risk of having babies with developmental problems, even without consuming alcohol. Alcohol, even in small amounts, blocks the ability of cholesterol to orchestrate the complex series of events involved in regulating cell fates and organ development in the embryo. Encouragingly, giving supplemental cholesterol to zebra fish embryos exposed to alcohol restored normal development.

Alcohol interferes with a precisely orchestrated biochemical signaling pathway that guides fetal development. Cholesterol is essential for a single pathway that governs the pattern of tissue development and it is vulnerable to the effects of alcohol. This new insight into the molecular basis of fetal alcohol syndrome could have far-reaching implications and suggests new prenatal care that might prevent the developmental defects caused by alcohol consumed during pregnancy.

### Adult Organ Rejuvenation

Giving alcoholics supplemental cholesterol may help slow down or prevent the occurrence of alcoholic liver disease, even chronic alcoholic induced cirrhosis, characterized by replacement of liver tissue by scar tissue, leading to progressive loss of liver function. The findings provide further credence to current practice of ensuring that pregnant women should not lower their cholesterol too low. A recent study found that women who took cholesterol-lowering drugs known as statins were at greater risk of giving birth to babies with developmental problems.

This new concept, stem cell nutrient and related technology, is also monumental for leading adult stem cell based healthcare and clinical practice in the coming years. This disclosed technology applied to adult health will have a major impact in anti-aging, organ and tissue regeneration, and prevention of alcohol related diseases.

Stem cell nutrients are foods that have both prescription and over the counter applicability. Thus the markets generally are:
Vitamin Supplements
Supplements aimed as Specific Organs
Nutritional Food Additive
Prescription Drug with a Variety of Delivery Methods
Supplement for Women subject to Pregnancy

The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements. In addition to the exemplary aspects and embodiments described above, further aspects and embodiments will become apparent by reference of the drawings and by study of the following descriptions.

### BRIEF DESCRIPTION OF THE SEVERAL VIEWS OF THE DRAWING(S)

The patent or application file contains at least one drawing executed in color. Copies of this patent or patent application publication with color drawings(s) will be provided by the Office upon request and payment of the necessary fee.

FIG. 1 graphically demonstrates dose-dependent effects of alcohol on the survival and phenotype of embryos.

FIG. 2 shows photocopies of the effects of fetal alcohol exposure which induces a phenotype spectrum similar to that of Hh-inhibited and cholesterol deficient embryos.

FIG. 3 demonstrates that alcohol exposure inhibits, Hh-signaling by decreasing the post-translational cholesterol modification of Shh.

FIG. 4 graphically demonstrates that alcohol exposures decrease cholesterol levels in embryos.

FIG. 5 shows that cholesterol supplementation rescues alcohol-induced embryo defects.

FIG. 6 shows Western blot analysis of Caveolin-1 and Shh distribution for protein lysates isolated from the rat hepatic stellate cell line HSC 8B.

FIG. 7 shows immunoprecipitation assays demonstrating decreases in Shh in Caveolin-1 caused by alcohol exposure.

FIG. 8 shows photocopies demonstrating the immunohistochemistry showing co-localization of Caveolin-1 and Shh in hepatic stellate cells.

FIG. 9 shows Western blot analysis of alcohol disturbing Shh co-localization with Caveolin-1 in lipid rafts and Shh accumulation in Golgi organelles.

FIG. 10 shows immunohistochemistry analysis of alcohol disrupting Shh entry into ER compartments and accumulation in Golgi organelles.

FIG. 11 demonstrates that alcohol exposure disrupts Shh secretion into the extra cellular matrix.

FIG. 12 shows merged fluorescent images of GFP specifically expressed in the liver of transgenic zebra fish.

FIG. 13 demonstrates thru cell cytometry is used to isolate GFP/Ptc+ cells from LFABP-GFP liver.

FIG. 14 shows cultured GFP/Ptc+ cells in an FGF/HGF hepatic inducing medium.

FIG. 15 shows that GFP-P/Ptc+ cells integrate into bile ductular epithelial cells and hepatocytes and begin to express GFP in pre-injured liver.

FIG. 16 shows that alcohol disrupts Shh protein in lipid graft co-localization.

FIG. 17 shows that alcohol exposures disrupt free cholesterol/cholesterol balance and transport in embryos.

FIG. 18 shows a Raman shift spectroscopy analysis which characterizes the alcohol and cholesterol signatures.

FIG. 19 demonstrates hedgehog activity in hepatic stellate cells.

FIG. 20 is a graphic demonstration of cholesterol derivative components preventing developmental defects.

FIG. 21 shows photocopies demonstrating that cholesterol and cholesterol-like molecules prevent alcohol induced embryonic developmental defects.

Exemplary embodiments are illustrated in reference figures of the drawings. It is intended that the embodiments and figures disclosed herein are to be considered to be illustrative rather than limiting.

### DETAILED DESCRIPTION OF THE INVENTION

### Cholesterol Treatment for Fetal Alcohol Syndrome

In this application, exposure of zebra fish embryos to low levels of alcohol during gastrulation blocks covalent modification of Sonic hedgehog by cholesterol. This leads to impaired Hh signal transduction and results in a dose-dependent spectrum of permanent developmental defects that closely resemble FASD. Furthermore, supplementing alcohol-exposed embryos with cholesterol rescues the loss of Shh signal transduction, and prevents embryos from developing FASD-like morphologic defects. Overall, a simple post-translational modification defect in a key morphogen may contribute to an environmentally induced complex congenital syndrome. This insight into FASD pathogenesis may suggest novel strategies for preventing these common congenital defects.

Post-translational protein modification plays an essential role in facilitating signal transduction regulation of gene expression. Protein modification by phosphorylation, acetylation, or methylation helps control the proper timing and sequence of events during embryogenesis; therefore, it is not surprising that defective modifications of these proteins can be important causes in the development of many types of congenital diseases. Accumulating evidence illustrates the importance of post-translational lipid modifications for regulating protein function. One example is the cholesterol and palinitoyl modification of Sonic hedgehog (Shh), which guides this protein's biogenesis, cellular trafficking, and functionality.¹

Shh is a highly conserved fetal morphogen that plays a central role in cell proliferation, differentiation, and embryonic patterning by activating the Hedgehog (Hh) signal pathway.^{2,3} The 45 kDa Shh precursor protein undergoes modification by auto-processing, followed by covalent linkage of cholesterol to the N-terminal proteolytic product.⁴ This mature, cholesterol-modified protein (19 kDa) can be transported to the cell membrane for secretion.⁵ Once secreted, the cholesterol-modified Shh ligand can initiate signal transduction by binding to its receptor, Patched (Ptc). Upon binding, Ptc relieves the inhibition of the signal transducer, Smoothened (Smo)⁶, which then activates Gli transcription factors by uncoupling them from the negative regulator, Suppressor of Fused.⁷ Gli is subsequently translocated to the nucleus and regulate expression of target genes including Ptc⁸, Gli1⁹ itself and NkX2.2.¹⁰

During embryogenesis, Shh is expressed specifically in Hensen's node, the floor plate of the neural tube, the cardiac mesenchyme, the early gut endoderm, the posterior portion of the limb buds, and throughout the notochord. As it is a morphogen, Shh also affects the development of tissues that are distal to where it is produced. Shh is apparently a key inductive signal for patterning of the ventral neural tube^{11,12}, the anterior-posterior limb axis¹³, and the ventral somites.¹⁴ In humans, one severe phenotype caused by mutations in Shh, or other components of Hh signaling, is holoprosencephaly (HPE)¹⁵, a disorder in which the fetal prosencephalon (forebrain) fails to divide to allow formation of bilateral cerebral hemispheres. HPE is also one of the extreme manifestations of severe fetal alcohol spectrum defects (FASD) in human embryos and in animal models of FASD¹⁵⁻²⁰. Similarly, production of Shh in the floor plate of the neural tube regulates the development of neural components in the overlying basal plate, including progenitors of motor neurons^{12,21}. FASD patients display delayed motor development and impaired fine- and gross-motor skills.²² Varying degrees of motor retardation have been observed in up to 89% of humans having FASD.²³⁻²⁵ Indeed, the diagnostic criteria for FASD include impaired fine motor skills.²⁶ Shh also has a proven role in neural crest morphogenesis²⁷, and FASD frequently includes defects in neural crest-derived structures. Clearly, there is significant overlap between the tissues affected by alcohol exposure, and those tissues that depend on Shh signaling for proper development.

Post-translational modification of Shh by cholesterol⁴ is a tightly regulated process that is necessary for the transportation and establishment of concentration gradients of the mature Shh ligand in developing embryos²⁸. Sterol- and fatty acid-modified Shh proteins form soluble multimers that are packaged in micelles for long-range transport.²⁹ Recent work has demonstrated that the activity and function of Shh protein varies significantly, depending upon the presence or absence of these modifications.⁵ The roles played by cholesterol-modified Still ligand in many facets of embryogenesis may account for some of the teratogenic effects of perturbed cholesterol biosynthesis in animal development.³⁰ Similar congenital defects occur in offspring of women who drink alcohol during pregnancy.

The teratogenic consequences of fetal exposure to alcohol are highly variable and include a spectrum of morphologic defects known as FASD.³¹ Phenotypic abnormalities of FASD include neurological, craniofacial, cardiac, and limb malformations, as well as generalized growth deficits and mental retardation.³² The mechanisms proposed to underlie the spectrum of birth defects caused by fetal alcohol exposure include: apoptosis³³, cell adhesion defects³⁴, accumulation of free radicals³⁵, dysregulation of growth factors³⁸, and altered retinoic acid biosynthesis³⁷. Some simple and essential questions have not been well explained by these hypotheses, for instance, how one or, at most, a few social drinks, cause fetal defects, why alcohol preferentially induces defects targeting some organs and tissues and not others, or why the pattern of defects seen in FASD is predictable and reproducible.

Alcohol can also impair prechordal plate migration³⁸ and disrupt the formation and function of Spernann's Organizer³⁹, a signaling center in gastrulating embryos that controls the patterning of the germ layers; the specific mechanisms that regulate axis pattern formation require highly evolutionarily conserved genetic pathways involving transcription regulatory circuitry and signal transduction pathways⁴⁰ Shh-containing vesicles contained within the organizer initiate a signal transduction pathway that plays a key role in embryonic patterning during development.⁴¹ Therefore, genetic or environmental factors that inhibit Shh signaling during gastrulation can disrupt proper patterning of the embryo. Interestingly, embryos that are exposed to alcohol during gastrulation³⁸ have defects that are similar to those found in embryos that have defects in Hh signal transduction⁴². A similar phenotype develops in embryos with a genetic defect in sterol homeostasis, for example, Smith-Lemli-Opitz Syndrome⁴³ (SLOS) or that are exposed to cholesterol lowering agents.^{44.45} These observations suggest that FASD may result from alcohol-dependent inhibition of cholesterol modification of Shh.

Many genetic disorders can result in abnormal regulation of cholesterol biosynthesis, storage, and trafficking. However, ethanol ingestion may be far more common mechanism for disrupting cholesterol homeostasis. Ethanol causes an inhibition of HMG-CoA reductase activity, which results in decreased free cholesterol in the cells, and reduction in circulating cholesterol levels.⁴⁶⁻⁴⁸ Acute ethanol exposure in perfused rat liver results in depletion of cholesterol in both liver homogenate and microsomes.⁴⁹ Ethanol specifically inhibits hepatic ACAT activity, which can lead to decreased cholesterol esters for transport in LDLS.⁵⁰ Thus, evidence from embryology, toxicology, and molecular biology indicates that a teratogenic mechanism underlying FASD links alcohol, cholesterol homeostasis, Shh signaling and cholesterol modification of functional Shh.

Several animal models have been to study FASD. The zebra fish model offers many advantages compared to insect and rodent models for alcohol and development studies: zebra fish are small in size, they have a large number of progeny, and they have rapid embryogenesis. This model has already been widely used in studies of developmental biology, genetics, gene function, signal transduction and high throughput drug screening. All of these characteristics make it an ideal model to delineate the molecular basis of the alcohol-induced birth defects.

It is shown that transient alcohol exposure during early development of zebra fish embryos causes dose-related inhibition of Hh signal transduction and produces a spectrum of permanent FASD-like defects. Alcohol-induced inhibition of Hh pathway activity parallels alcohol disruption of cholesterol homeostasis and decreased cholesterol-modification of the Shh ligand. Supplementing the alcohol-exposed embryos with cholesterol rescues the loss of Shll signal transduction, and prevents embryos from developing FASD-like morphologic defects.

### Materials and Methods

### Alcohol, Cyclopamine, and AY-9944 Treatment

Embryonic alcohol exposures were adapted from a previous report.³⁸ Embryos with chorions were exposed to six different concentrations of alcohol (eg, 0, 0.25, 0.5, 1.0, 1.5, and 2.0% (v v⁻¹)) in embryo medium. Embryos in sealed Petri dishes were exposed to alcohol for 6 h beginning at the dome stage (ie, 4.25 hours post-fertilization (hpf) or 30% epiboly stage) and were incubated at 28.5° C. Immediately following alcohol exposure, embryos were harvested for analysis of Hh pathway activity, cholesterol content, or tissue alcohol concentration. The remaining embryos were washed and incubated in alcohol-free medium. Embryos were then harvested at 1, 2, or 4 days post-fertilization for survival and phenotypic analyses. Cyclopamine (11-deoxojervine) is a naturally occurring chemical that inhibits the Hh signaling pathway by functioning as an antagonist of smoothened protein. AY9944, trans-1,4 bis-(2-dichlorobenzylaminomethyl) cyclohexane dihydrochloride blocks cholesterol synthesis through inhibition of 7-dehydrocholesterol reductase. AY-9944 (7.5 μM, Sigma-Aldrich) and cycloparnine (10.0 μM, Calbiochenn) were administered in the same manner as alcohol. Following treatment, embryos were washed and incubated in normal medium for up to 4 days post-fertilization.

### Alcian Blue Staining and Immunohistochemistry

Staining for skeletal structures was performed as previously described.⁵¹ Immunohistochemistry is carried out with following primary antibodies (Hybridoma Bank, 1:10): MF20 to stain myocardium and facial muscles, S46 to identify ventricular myocardium, and F59 to identify slow muscle progenitors in the somites. The secondary antibodies were Alexa 568-conjugated goat anti-mouse IgG_{2g} and Alexa 488-conjugated goat anti-mouse IgG_{2g} (1:400, Molecular Probes). The embryos were mounted and imaged.

### Measurement of Cholesterol Content

Following alcohol exposure, lipids were extracted from embryos (n≧14) with chloroform-methanol (2:1) in duplicate. Cholesterol content was then measured by the Arnplex Red Cholesterol Assay Kit (Invitrogen).

### Measurement of Tissue Alcohol Concentration

Following alcohol exposure, embryos (n=38) were pooled from each treatment group in triplicate. An Alcohol Test Kit (Diagnostic Chemicals Limited) was used to determine the tissue alcohol concentrations in treated embryos.

### RT-PCR and Real-Time Quantitative Analysis

Total RNA was extracted from embryos (n=10) with RNeasy kits (Qiagen). RT-PCR were performed using primers (information listed in following table) as previously described.⁵²

PRIMERS INFORMATION TABLE

| | | | | |
|---|---|---|---|---|
| Gapdh | AY | Forward | 5′ ACTCCACTCATGGCCGTTAC | 262 |
| | 818346 | Reverse | 5′ TGGTTGACTCCCATCACAAA | |
| Shh | NM | Forward | 5′ TCTCGCATTAAGTGGCTGTG | 397 |
| | 131063 | Reverse | 5′ GCTTGTAGGCGAGAGGTGTC | |
| Ptc | NM | Forward | 5′ CATCCCATTCAAGGAGAGGA | 282 |
| | 130988 | Reverse | 5′ TGAATGACCCGAGATGAACA | |
| Gli1 | NM | Forward | 5′ GGGAGCGCCAATAATAATGA | 350 |
| | 178296 | Reverse | 5′ CATTGGCCTGAAGTGTTGAA | |
| Nkx2.2 | NM | Forward | 5′ CGTATAGCGCCCAGTCTCTC | 219 |
| | 131422 | Reverse | 5′ AGAAAGGGTCAAGCTGCAAA | |

### Immunoblot

Total cellular protein was isolated as previously described⁵¹ and cell membrane proteins were isolated using the Mem-PER(r) Eukaryotic Membrane Protein Extraction Reagent Kit (Pierce Biotechnology). Proteins (4o μg) in Laemmli buffer were then separated by 12% Tris-HCl SDS-polyacrylamide gel electrophoresis and transferred to a PVDF membrane. Membranes were blocked, washed, and exposed to primary antibodies (Santa Cruz Biotechnology) against Shh (N-19, Catalog number: sc-1194; dilution: 1:2.500) and β-actin (1:1.000). Signals were detected by Anti-goat HRP antibody (1:10.000, Amersham).

### Gli-Luciferase Reporter Assay

The Gli-luciferase reporter assay was performed in replicate experiments of pooled embryos (n=15). Briefly, zebra fish embryos at the 1-2 cell stage were microinjected with 0.5 nl of Gli-BS-Firefly luciferase plasmid (60 ng nl⁻¹) and Renilla luciferase plasmid (60 ng nl⁻¹, pRL-TK, Promega). Reporter activity was determined by using the Dual-Luciferase Reporter Assay System (Promega). Activity of the Firefly luciferase reporter was normalized to the activity of a Renilla luciferase internal control for transfection efficiency.

### Cholesterol Microinjection

Embryos were microinjected at 1-2 cell stage with 0.2 nl of 5 μg μl⁻¹ (10 pg) cholesterol (BioVision Inc.) with or without the two plasmids for the Gli-luciferase reporter assay. Embryos were allowed to develop and were then treated with alcohol as previously described. At 48 hpf embryos were analyzed.

### Results

### Alcohol Exerts Teratogenic Effects in a Dose and Stage-Specific Manner

The zebra fish model was chosen to evaluate the hypothesis because it permits exposure to precise concentrations of alcohol during well-defined developmental time frames. Zebra fish embryos were exposed to a range of alcohol concentrations (0, 0.25, 0.5, 1.0, 1.5, and 2.0% v/v in embryo medium at two different time windows during development. The first exposure window occurs from 1 to 2 cell stages to 3 hours post fertilization (hpf), and the second exposure window occurs between 4.25 and 10.25 hpf during the late blastula stage and the gastrula stage. Exposure to alcohol during the first exposure window is almost uniformly fatal. Fewer than 40% of the 897 embryos from this time frame that were treated with the lowest alcohol concentration (0.25%) survived to 48 hpf.

Embryos exposed to alcohol during the second exposure window had much better survival rates than those exposed during the zygote stage to the same levels of alcohol. During the late blastula-gastrula stage, survival of the exposed embryos was also dose dependent. For example, 10% of 202 embryos exposed to 3% alcohol during this time frame were alive at 48 hpf, compared to a survival rate of over 90% for the 897 embryos exposed to 0.25% alcohol for 6 h during the same developmental time frame. A more detailed analysis was performed at 48 hpf by scoring alcohol effects in three categories: (a) dead, (b) alive with abnormal phenotype, or (c) alive without abnormal phenotype.

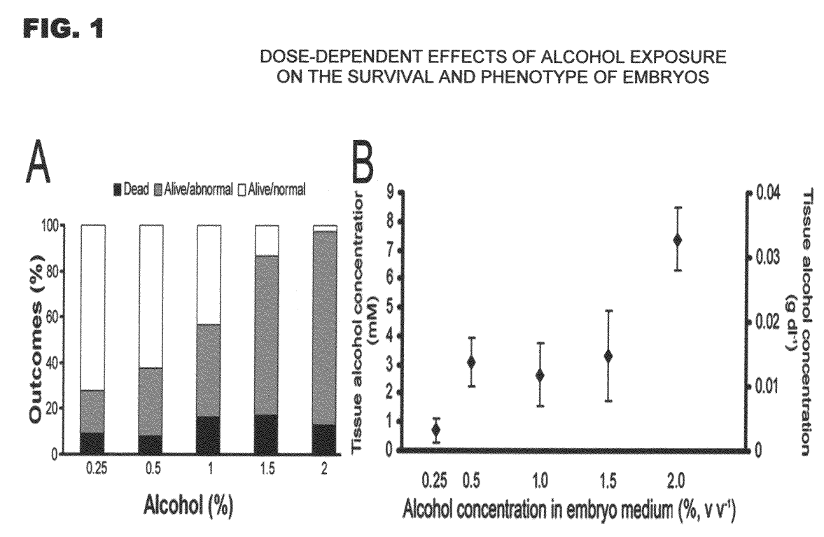

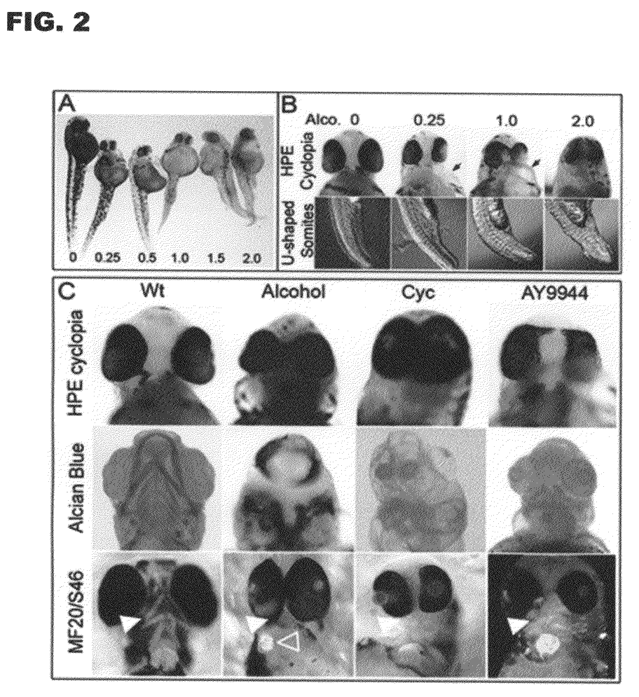

As shown in FIG. 1A, during the late blastula-gastrula stage, the phenotype at 48 hpf depended upon the dose of alcohol that embryos were exposed to. For example, 84% of the embryos exposed to 2% alcohol during the second exposure window survived through 48 hpf and exhibited abnormal morphology, while only 2.6% of the living embryos were phenotypically normal. (See FIG. 2 for illustrations of representative defects). This level of exposure was lethal for the remaining 13% of embryos. In contrast, 18% of the embryos exposed to 0.25% alcohol during the second exposure window were alive and had minimal abnormalities at 48 hpf; the majority (72.3%) were alive and had normal phenotypes and <10% failed to survive to the 48 hpf time point. The frequency of these alcohol-induced phenotypes has been characterized in Table 1.

TABLE 1
Frequency of Phenotypes in Alcohol-Treated Embryos

| | | | | | | Defected |
|---|---|---|---|---|---|---|
| | | | | Heart | | Somites |
| Alcohol | | | Cyclopia | edema | Arch bone | shortened |
| (%)^{a} | n | HPE (%) | (%)^{b} | (%) | defect (%) | tail (%) |
| 0 | 147 | 0 | 0 | 0 | 0 | 0 |
| 0.25 | 116 | 0.86 | 0.86 | 0.86 | 1.724 | 1.72 |
| 0.5 | 123 | 1.62 | 0 | 9.76 | 1.626 | 1.63 |
| 1.0 | 115 | 6.96 | 10.43 | 17.39 | 17.39 | 17.39 |
| 1.5 | 92 | 28.26 | 27.17 | 28.26 | 33.70 | 40.22 |
| 2.0 | 110 | 59.09 | 57.27 | 66.36 | 57.27 | 60.00 |

^{a}Alcohol concentration in embryos medium at v/v (%), embryos were treated at the dome/30% epiboly stage (4.3 hpf) for 6 h, phenotype were analyzed at 48 hpf for HPE, cyclopia and heart edema. Defective arch bones and shortened tails were analyzed at day 4 after fertilization by alcian blue staining.
^{b}Cyclopie phenotypes include full and partial cyclopia that with seperate, but more closely spaced eyes.

As shown in FIG. 2, fetal alcohol exposure induces a phenotype spectrum similar to that of Hh-inhibited and cholesterol deficient embryos. (a) Increasing alcohol exposures cause cranial-to-caudal defects, including generalized growth retardation in 2-day-old embryos. (b) Alcohol induces HPE, cyclopia, pericardial edema (arrow), and U-shaped somites. (c) Side-by-side comparison of 4-day-old untreated (wild-type, Wt), alcohol-exposed (2%), cyclopamine-treated (Cyc, lo i, M), and AY-9944-treated (7.5 uM) embryos show shared developmental malformations including HPE and cyclopia, underdeveloped of the craniofacial bones (Alcian blue)/muscles (arrowhead, MF20, red), and failure of the heart tube to loop (open arrowhead, myocardium, MF20 (red), ventricular myocardium, S46 (green/yellow)).

To assure that the alcohol-induced morphologic defects seen in our model do not merely reflect exposure to supraphysiologic concentrations of alcohol, applicant measured the alcohol concentrations in fetal tissue following alcohol exposures. Tissue alcohol concentrations in zebra fish embryos correspond to the exposed alcohol concentration in embryo medium 0.25-2.0% range from 0.71-7.4 mM or 0.003-0.034 g dl⁻¹. (FIG. 1B) These alcohol concentrations can be achieved in the blood of a human being by consumption of one or, at most, a few social drinks. As shown in FIG. 1, embryos (n>64) were exposed to increasing concentrations of alcohol and evaluated at 48 hpf. Embryos were scored as alive/normal, alive/abnormal, or dead. These three categories are expressed as a percentage of the total number of embryos in each cohort. Tissue alcohol concentrations in zebra fish are related to the level of alcohol exposure. In general, the alcohol concentrations were in the range of 0.71-7.4 mM or 0.003-0.034 g dl-⁻¹. Error bars indicate 1 s.e.m. of three experiments (n=38 fish per group).

### Morphological Defects Induced by Fetal Alcohol Exposure Recapitulate the Development Abnormalities with Hh Signaling Defects

Alcohol-induced defects in zebra fish embryos recapitulated those that occurred in other species following fetal alcohol exposure. In a dose-dependent fashion, transient alcohol exposure for 6 h during gastrulation resulted in subsequent permanent phenotypic abnormalities while only a modest increase in the rate of embryo mortality was observed (FIG. 1A). By 48 hpf, a cumulative cranial-to-caudal phenotype was evident in embryos that were exposed to alcohol transiently during gastrulation (FIG. 2).

These embryos were growth retarded (FIG. 2A), and exhibited a dose-dependent spectrum of phenotypes that included neurological, craniofacial, cardiac, and body axis defects. Embryos exposed to the highest alcohol concentrations had overt HPE, cyclopia (complete or partial), pericardial edema, U-shaped somites and severely foreshortened tails (FIGS. 2A and 2B). Comparisons performed between alcohol-treated embryos and embryos transiently treated with cyclopamine, a specific inhibitor of Hh signaling, or AY-9944, an inhibitor of cholesterol biosynthesis and transportation, during gastrulation revealed that embryos from all three groups had HPE and partial cyclopia, as well as underdevelopment of the craniofacial bones and muscles, and failure of the heart tube to loop (FIG. 2C). These observations support a role for defective Shh signaling in the pathogenesis of FASD and are consistent with the possibility that alcohol inhibits Shh activity by interfering with cholesterol modification of the cleaved 19 kDa protein.

### Alcohol Disrupts Hh Pathway Activity without Significant Change of Shh Protein Expression, but does Decrease Levels of Cholesterol-Modified Shh

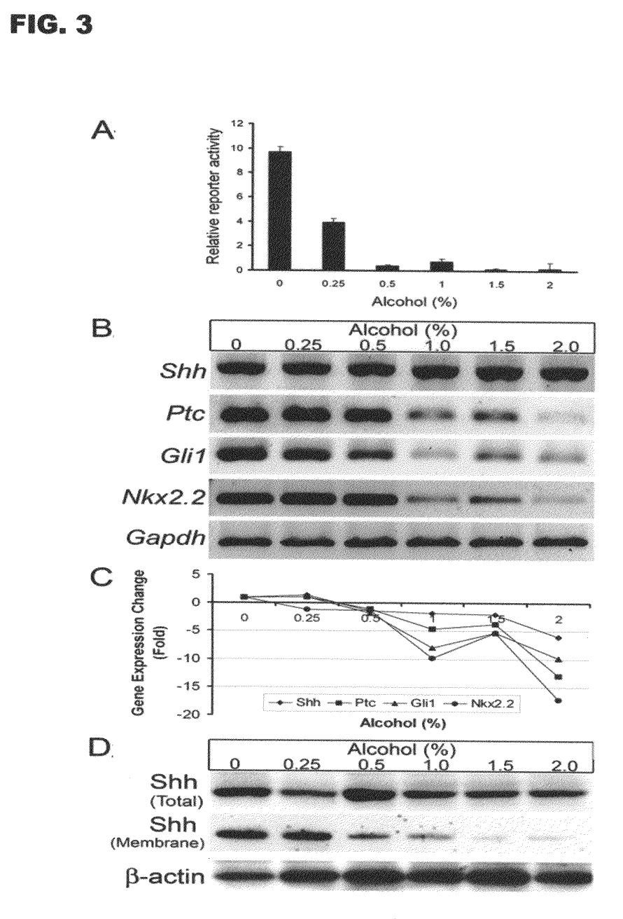

To investigate this further, Shh signaling activity was directly measured by examining developing embryos that were microinjected with a Shh-responsive, Gli-BS luciferase reporter construct at the 1-2 cell stage⁵³. Following alcohol exposure, both luciferase activity (FIG. 3A) and the expression of Shh-regulated genes, such as Ptc, Gli1, and Nkx2.2 (FIG. 3B), decreased in a dose-related fashion. Real-time quantitative PCR analysis confirmed that there is a threshold for alcohol-induced inhibition of the expression of Shh target genes. While exposure to a very low alcohol concentration (0.25%) caused no significant change in the expression of Ptc or Gli1, Nkx2.2 expression decreased by about 50% under these conditions. In response to a 0.5% alcohol treatment, expression of these three Shh target genes decreased by from 1.3 to 1.9 fold, and alcohol concentrations ranging from 1.0 to 2.0% caused expression of those genes to decrease from 5 to 17 fold (FIG. 3C). In contrast to the results obtained by RT-PCR, real-time quantitative PCR showed that Shh transcription decreased when embryos were exposed to alcohol concentrations higher than 1.0%. Notably, inhibition of Shh signaling occurred despite relatively stable levels of Shh protein from whole cell lysates (cytosolic and membrane). However, inhibition of Shh signaling was associated with a progressive loss of Shh from the cellular membrane protein fraction isolated from FASD embryos (FIG. 3D). Given that Shh must be covalently modified by cholesterol to anchor in plasma membranes, these results suggest that cholesterol modification of Shh may be impaired in alcohol-treated embryos.

Alcohol exposures inhibit Hh signaling by decreasing the post-translational cholesterol modification of Shh. (a) As seen in FIG. 3, dose-related reduction of a Hh-responsive Gli-luciferase activity (normalized by Renilla luciferase) in alcohol-exposed embryos. Error bars indicate 1 s.e.m. of four replicate experiments. (b) RT-PCR analysis of gene expression levels of Shh, and its target genes, Ptc, Gli1, and Nkx2.2, as Well as the GAPDH housekeeping gene following alcohol exposure. (c) Semi-quantitative expression analysis of Shh and its target genes by real-time RT-PCR, data is normalized to the internal control of GAPDH. (d) Western blot of the total (cytosolic and membrane) and membrane fractions of Shh protein from alcohol-exposed embryos. β-Actin was employed as the loading control.

### Alcohol Treatment Alters Cholesterol Homeostasis and Reduced Cholesterol Ester Content

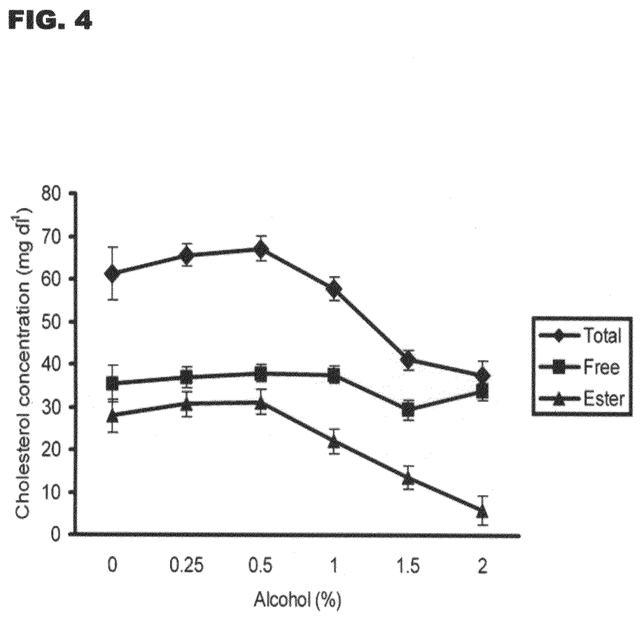

As shown in FIG. 3, alcohol exposure during the late blastula-gastrula stage causes a dose-dependent reduction in membrane associated Shh. Given that esterification of Shh by cholesterol drives its membrane localization, these results also suggest that alcohol exposure reduces cholesterol ester formation. Applicant tested whether alcohol exposure impairs general sterol homeostasis during gastrulation by measuring cholesterol levels in whole embryo extracts. In a dose-related fashion, alcohol exposure resulted in a decrease in the total cholesterol content of the embryos (FIG. 4). As seen in FIG. 4, alcohol exposures decrease cholesterol levels in embryos. Dose-related reduction of total cholesterol and cholesterol ester levels in alcohol-exposed embryos. Error bars indicate 1 s.d. This was mostly due a reduction in total cholesterol esters that paralleled the dose-dependent decreases in cholesterol-modified Shh protein and Shh signaling activity (FIGS. 2A and 2B), and correlated with a dose-dependent acquisition of alcohol-induced morphologic defects: (FIG. 2C) the higher the dose the more severe and extensive the defects. Together with evidence that AY-9944 lowers cholesterol and produces similar defects, our findings suggest that alcohol interrupts cholesterol homeostasis and that depleted stores of cholesterol results in impaired cholesterol modification of Shh, leading to decreased Shh signaling, which causes a FASD-like phenotype.

Alcohol exposures decrease cholesterol levels in embryos. Dose-related reduction of total cholesterol and cholesterol ester levels in alcohol-exposed embryos.

### Supplementation of Cholesterol Rescues Hh Signaling Defects Caused by Alcohol and Prevents Fetal Alcohol Induced Developmental Defects

To confirm the importance of this potential molecular mechanism for FASD, applicant performed rescue experiments in alcohol-exposed embryos. Supplemental cholesterol, Gli-BS-Firefly luciferase plasmid, and Renilla luciferase plasmid were co-microinjected into 1-2 cell stage embryos, which were then treated with various alcohol concentrations during gastrulation.

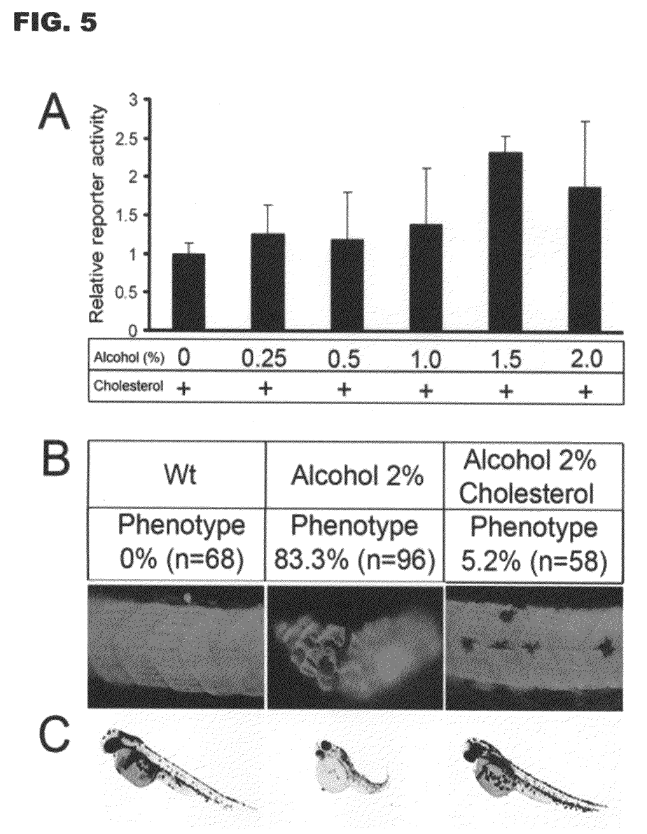

Subsequent studies showed that Gli-BS reporter activity was preserved at all doses of alcohol exposure in cholesterol-supplemented embryos (FIG. 5A). To determine whether recovery of Shh activity leads to the rescue of Hh-dependent cell differentiation, applicant studied the slow muscle pioneer (MP) cells in somites⁴² using the F59 antibody which specifically identifies these progenitors.⁵⁴ In untreated embryos, at 48 hpf, applicant observed an organized, V-shaped pattern of MP fibers with approximately 20 fibers per somite pair. Microinjection of 5% DMSO (the cholesterol dissolving vehicle) had no adverse effect on zebra fish development. In contrast, alcohol-exposed embryos had a disorganized, diffuse pattern of MP fibers at this time. The cholesterol supplemented, alcohol-exposed embryos had a similar number and pattern of MP fibers as the untreated, wild-type embryos (FIG. 5B). Furthermore, most (94.8%) of the cholesterol-supplemented, alcohol-exposed embryos (n=58) appeared grossly normal, unlike 83.3% of the alcohol-exposed, unsupplemented embryos (n=96) that had FASD-like phenotypes (FIG. 5C). Thus, cholesterol supplementation rescues alcohol inhibited Shh signaling, and prevents alcohol-induced defects at the molecular, cellular, and developmental levels.

Cholesterol supplementation rescues alcohol-induced defects. (a) Rescue of Hh-responsive Gli-BS-luciferase reporter activity (normalized by Renilla luciferase) in alcohol-exposed embryos with cholesterol supplementation. Error bars indicate 1 s.e.m. of 11 replicate experiments. (b) Shown are untreated (Wt), alcohol-exposed (2%), and cholesterol-supplemented (10 pg), alcohol exposed (2%) embryos. F59 staining of slow muscle fibers (green). (c) Lateral views of 48 hpf zebra fish embryos demonstrate morphologic rescue in the cholesterol-supplemented, alcohol-exposed embryos.

### Discussion

In a number of previous studies, researchers have used zebra fish to determine alcohol-related effects on development.⁵⁵⁻⁵⁹ Applicant has extended these results by using this model to identify a novel molecular mechanism that may be responsible for alcohol's teratogenic effects, namely, alcohol-induced inhibition of the cholesterol modification of Sbh, which subsequently inhibits Shh signal transduction; inhibition of this pathway appears to play the key role in the development of FASD pathogenesis.

As zebra fish lack placentas and develop ex utero, and alcohol dehydrogenases^{60,61} are not expressed in embryos at the time exposed to alcohol (ie from 4-10 hpf), Thus, the metabolites generated by oxidation of ethanol are not likely to be a major cause of the induced phenotypes. Even at very low tissue concentration, alcohol may directly causes developmental defects, instead of alcoholic metabolites from maternal resource or the embryo.

Direct measurements determined a range of alcohol concentration from 0.71-7.4 mM or 0.003-0.034 g dl⁻¹ in fetal tissue under our experimental conditions. These alcohol concentrations are about 5.9- to 123-fold lower than blood alcohol levels that induce FASD in mice⁶²; these concentrations are also 4.2- to 153-fold lower than the alcohol concentrations that induce cell apoptosis⁶³, and retinoic acid deficiency³⁷ or that have antagonistic effects on growth factors³⁶. Hence, relatively low concentrations of fetal tissue alcohol also can induce FASD-like defects. Blood alcohol concentrations in this range are achieved in a 55-kg female following the consumption of one 12-ounce beer. This may explain why alcohol is the most common teratogen responsible for human congenital defects, and suggests that there is no safe level of alcohol consumption during pregnancy.

Fetal alcohol exposure impairs hedgehog cholesterol modification and signaling. The morphological phenotypes induced by alcohol in zebra fish recapitulate the FASD defects seen in other species. For instance, similar defects have also been reported in human embryos that have FASD^{23,64} microcephaly in 84%, eye problems in 25%, cardiac developmental defects in 29%, various problems with truncal muscles and bones, including slack muscles in 58%, swallowing/feeding problems in 20%, hip deformities in 9%, pidgeon chest in 30%, concave chest in 7%, and spinal dimple in 44%. FASD patients also have craniofacial abnormalities such as facial anomalies (95%), small teeth (16%), cleft palate (7%), and overall growth retardation (98%). Thus, it seems that the ethanol-related developmental defects that applicant has observed in zebra fish embryos nicely parallel those observed in humans having FAS.

The evidence presented here and elsewhere^{39,65} consistently demonstrates that fetal alcohol exposure inhibits the transcription of Shh responsive genes. Notably, applicant found that total Shh protein level in the zebra fish embryonic tissues was not significantly changed by any of the tested alcohol exposures. However, the treated embryos exhibited FASD-like phenotypes and unpaired Hh signal transduction, suggesting that defective Shh signaling is the key factor in the morphological defects induced by alcohol. Furthermore, applicant has now shown that the defect in Shh signal transduction is due to disruption of the post-translational cholesterol modification of Shh. These findings help to explain why over-expression of Shh mRNA alone does not consistently rescue alcohol-induced morphologic defects or the decrease in expression of Shh responsive genes. ^{38,65}

Here, applicant observed that supplementing a simple chemical, cholesterol rescues the alcohol-inhibited Hh signal transduction and prevents embryos from developing FASD-like morphologic defects. Recently, sterols were shown to directly activate the Shh signaling pathway through Smo. Cholesterol, or certain oxyterols, serve as Smo agonists.⁶⁶ This evidence suggests that cholesterol may also directly stimulate Smo to initiate Shh signal transduction, bypassing the pathway and rescuing alcohol-induced Shh dependent development. It will be interesting to determine whether the same cholesterol supplementation strategy that rescues alcohol-exposed embryos might protect embryos from tetratogenicity caused by other environmental factors that induce cholesterol-related defects in Shh signaling.

The mature Shh peptide is doubly lipid-modified, having a cholesterol moiety at its C terminus⁴ and a palmitate attachment at Cys-24 of the N terminus.⁶⁷ The N-terminal lipid is required for inducing the differentiation of ventral forebrain neurons.⁶⁸ In contrast, in the absence of the N-lipid, the C-terminal lipid-containing Shh is sufficient to induce mouse digit duplication.⁶⁹ Mouse mutants have been created in which Shh lacks cholesterol modification, lacks palmitoylation, or lacks both types of lipid modification. Functional analysis of these mutants clearly demonstrated that both types of lipid modification are essential for regulating the range and shape of the Shh morphogen gradient during early development.^{70.71} For future direction, it remains to be determined whether the alcohol-induced defect in cholesterol modification influences N-terminal palmitoylation, or Shh cellular trafficking in lipid rafts, or affects the binding affinity of Shh to Ptc, or even the gradient shape and content of Shh.

Overall, applicant has shown that a simple post-translational modification defect of a key morphogen results in a complex congenital disease. This new insight into the molecular basis of FASD has far-reaching implications, and suggests novel prenatal interventions that might prevent FASD developmental defects.

### Caveolin-1 Binds with Shh to Form a Protein Complex

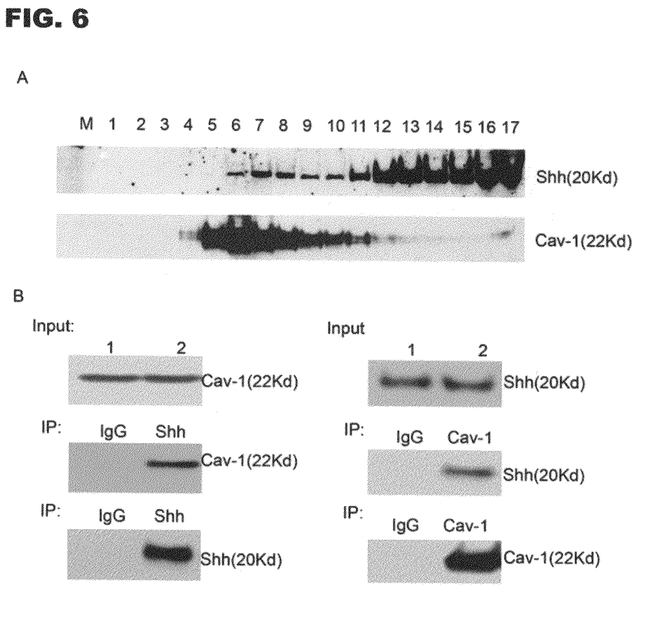

Applicant has observed that alcohol exposure results in defective Shh-cholesterol modification and impairs the accrual of plasma membrane associated Shh, suggesting that alcohol causes a defect in the intracellular transport of this ligand (Li, 2007). In order to obtain detailed information on the distribution of Shh within cells, and as a first step toward determining the mechanisms underlying Shh intracellular trafficking, applicant used non-ionic detergent protein extraction and density gradient ultracentrifugation to fractionalize and isolate cellular proteins. After ultracentrifugation, applicant collected 17 individual gradient fractions (each fraction contained 500 μl), from the lowest density (at the top of the centrifuge tube) to the highest (at the bottom of the tube). The distribution of lipid raft proteins was primarily confined to fractions 4 through 11, as indicated by the presence of the lipid raft-associated protein, Caveolin-1 (FIG. 6A, bottom panel). Applicant also found that the distribution of Shh was confined to fractions 6 through 17; Shh co-localized with Caveolin-1 in fractions 6 through 11 generated by the density gradient ultracentrifugation (FIG. 6A, top panel).

FIG. 6 shows representative western blot analysis of Caveolin-1 and Shh distribution in density gradient ultracentrifugation fractions for protein lysates isolated from the rat hepatic stellate cell line HSC 8B (A). immunoprecipation assays demonstrate that Shh and Caveolin-1 physically interact to form a protein complex (B, C). Equal amounts of cell lysates were used in the immunoprecipitation assays and expression levels of the target proteins were confirmed by Western blot analyses using either anti-Caveolin-1 antibody (B, top pane, line 2) or anti-Shh antibody (C top panel, line 2). Both Caveolin-1 (B, middle panel and B bottom panel) and Shh (B bottom panel and C middle panel) were detected in both anti-Caveolin-1 and anti-Shh antibody immunoprecipitates, but not in the IgG negative control precipitates (line 1, middle and bottom B and C).

Physical co-localization of Shh and Caveolin-1 in the density gradient may suggest that these two proteins have same similar physical characteristics or that they functionally interact, however, it does not necessarily indicate a direct physical interaction between them. Since Caveolin-1 plays an important role in protein and cholesterol transport and trafficking, a direct interaction between Caveolin-1 and Shh is an intriguing possibility. In order to establish whether these proteins physically interact to form a protein complex, applicant performed a series of immunoprecipitation assays on total protein isolated from the rat hepatic stellate cell line, HSC8B, which expresses high levels of Shh. To determine whether there is a direct physical interaction between these two proteins, applicant used an anti-Caveolin-1 antibody in an immunoprecipitation assay of HSC8B cell lysates, followed by Western blots analyses using anti-Caveolin-1 and anti-Shh antibodies. Applicant confirmed equal loading of the precipitated proteins using the anti-Caveolin-1 antibody (FIG. 6A, top panel). As expected, the anti-Caveolin-1 antibody precipitated the 22 kDa Caveolin-1 protein (FIG. 6B, middle panel, line 2). Furthermore, it precipitated the mature 20 kDa Shh ligand (FIG. 6B, bottom panel, line 2), indicating that Caveolin-1 physically associates with Shh. In the negative control, IgG antibody was unable to precipitate either Caveolin-1 or Shh (FIG. 6, line 1, middle and bottom panel). Moreover, applicant confirmed the interaction between these two proteins by using an anti-Shh antibody in an immunoprecipitation assay of the HSC8B cell lysate. The conditions used in this experiment paralleled those used in the previous immunoprecipitation assay; in this case, equal protein loading was confirmed using the anti-Shh antibody (FIG. 6C, top panel). Applicant found that both Shh (FIG. 6C, middle panel) and Caveolin-1 (FIG. 6C, bottom panel) are immunoprecipitated from HSC8B cell lysates by the anti-Shh antibody. The fact that anti-Shh and anti-Caveolin-1 antibodies each co-precipitate Caveolin-1 and Shh from HSC8B cell lysates strongly suggests that Caveolin-1 interacts with Shh to form a protein complex in this Shh producing cell line.

### Alcohol Specifically Decreases the Formation of the Caveolin-1/Shh Complex in Lipid Rafts

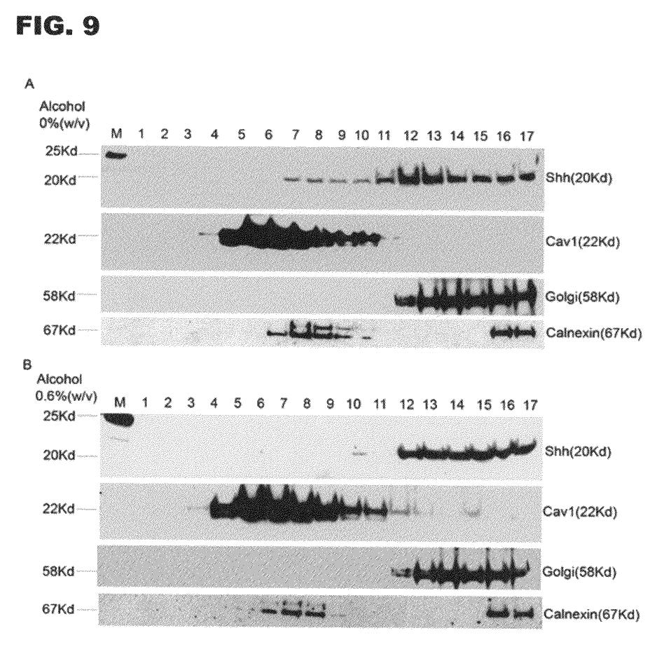

The observation that Shh directly interacts with the lipid raft protein Caveolin-1 raises an interesting question regarding previous work in which applicant showed that alcohol exposure does not affect total Shh levels, but instead results in defective Hh signal transduction by causing a dose-dependent decrease in the concentration of cholesterol-modified, mature Shh ligand at cell plasma membranes (Li, 2007). To determine whether this decrease in plasma membrane-associated Shh ligand is specifically linked to the interaction between Caveolin-1 and Shh in lipid raft microdomains, applicant investigated the affect of alcohol exposure on the co-localization of Caveolin-1 and Shh in density gradient fractions. First, as described in FIG. 6, cellular proteins isolated by non-ionic detergent resistant cellular extraction were fractionalized by density gradient ultracentrifugation. Lipid raft associated proteins were present in fractions 4 through 11, as indicated by the presence of Caveolin-1 (FIG. 6A, middle panel and FIG. 9A, second panel); applicant specifically used fractions containing lipid raft associated proteins (fractions 7-9) for use in immunoprecipitation assays with an anti-Caveolin-1 antibody. HSC8B cells were exposed to various concentrations of alcohol (0, 0.3, 0.5, 0.6, and 0.8% w/v corresponding to 0, 55, 81, 109 and 136 mM) for two hours prior to protein extraction and density gradient ultracentrifugation. Fractions 7 through 9 from the density gradient were pooled and used in the immunoprecipitation assay.

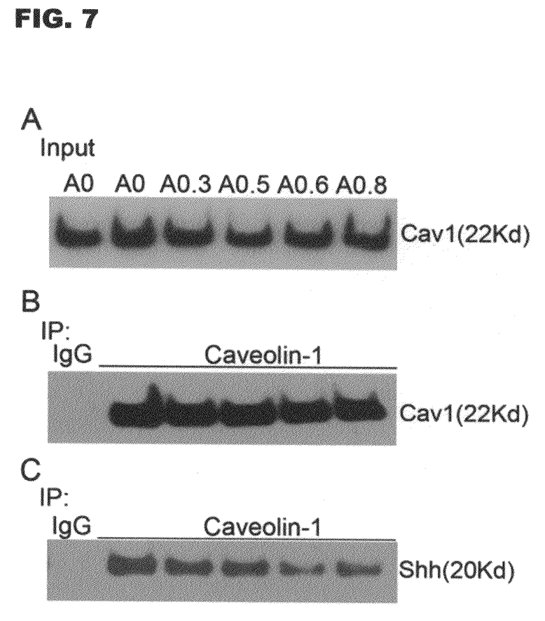

Equal loading of proteins for use in these assays was confirmed by Western blot analysis using the anti-Caveolin-1 antibody (FIG. 7A). In these immunoprecipitation assays, applicant determined that the amount of Shh found in the Caveolin-1 containing lipid raft fractions (fractions 7-9) decreased in an alcohol dose-dependent manner (FIG. 7C); the alcohol treatments did not affect the amount of Caveolin-1 in the lipid raft fractions (FIG. 7B). IgG antibody, which was used in negative control immunoprecipitation assays, did not precipitate either Caveolin-1 or Shh. In FIG. 7, Immunoprecipitation assays demonstrate that alcohol exposure decreases the amount of Shh in Caveolin-1/Shh complexes located in lipid raft fractions. Using non-ionic detergent resistant protein extraction, followed by sucrose gradient centrifugation, lipid rafts and their associated proteins were isolated from HSC 8B cells treated with various alcohol concentrations (alcohol concentration w/v: 0.3%, 0.5%, 0.6% and 0.8% for 2 hours) and from untreated cells. Anti-Caveolin-1 antibody was used to immunoprecipite proteins from lipid raft preparations; equal amounts of protein were ensured by Western blot analysis using the anti-Caveolin-1 antibody (A), and the immunoprecipitates were probed with both anti-Caveolin-1 (B) or anti-Shh antibodies (C). The amount of Caveolin-1 protein in the lipid rafts was not affected by alcohol exposure (B); however, alcohol exposure decreased the amount of Caveolin-1-associated Shh in a dose-depended manner (C).

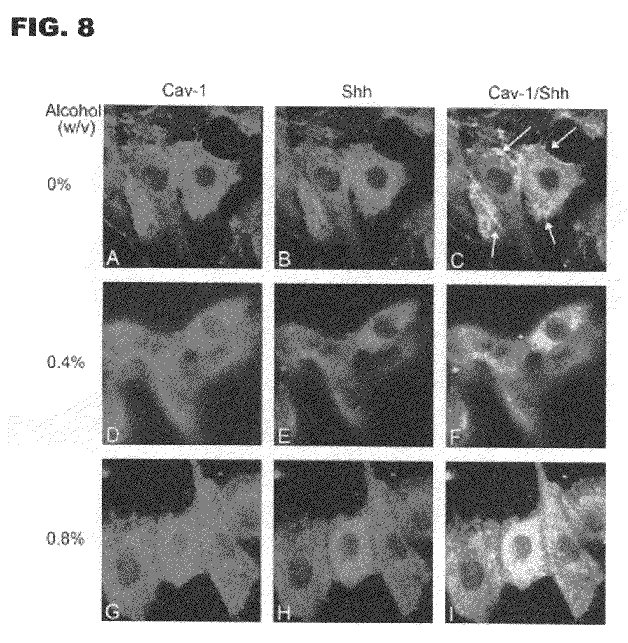

We also used immunohistochemistry to evaluate the effect of alcohol on Caveolin-1/Shh complex formation. In FIG. 8, immunohistochemistry revealed co-localization of Caveolin-1 and Shh. In untreated hepatic stellate cells, Caveolin-1 (A, green) and Shh (B, red) co-localized in the cytoplasm, and particularly in the cell plasma membrane (C, yellow, indicated by arrows). When cells were exposed to 0.6% (w/v) alcohol for 30 minutes, Shh levels were not affected; however, the amount of Shh co-localizing with Caveolin-1 at the plasma membrane dramatically decreased. Double-staining HSC 8B cells with anti-Caveolin-1 antibodies (FIG. 8A, green) and anti-Shh antibodies (FIG. 8B, red) revealed a punctate, salt-and-pepper, co-localized distribution of Shh and Caveolin-1 in the cytoplasm, particularly at the plasma membranes (FIG. 8C, yellow as indicated by arrows). Exposure of HSC 8B cells to 0.4% (w/v) (FIGS. 8D-F) and 0.8% (w/v) (FIG. 8G-I) alcohol for thirty minutes did not produce noticeable changes in either Caveolin-1 (FIGS. 8D and 8G, green) or Shh (FIGS. 8E and 8H, red) levels; however, the amount of punctate co-localized particles dramatically decreased in an alcohol dose dependent manner, nearly disappearing from the cell plasma membranes at high alcohol concentrations (FIGS. 8F and 8I, yellow). Both biochemical and immunohistochemical analyses indicated that Caveolin-1 and Shh form a protein complex, and showed that this complex accumulates in a lipid raft domains in plasma membranes. Alcohol exposure disrupts the formation of the Caveolin-1/Shh complex and leads to decreased levels of Shh in plasma membranes, particularly in the lipid raft structures. These observations suggest that alcohol exposure causes a defect in the secretion and transport of the Shh ligand.

### Alcohol Exposure Causes Defective Transport of Shh into the ER and Leads to Accumulation in the Golgi Compartment

In order to begin delineating the detailed molecular mechanisms that underlie the deleterious effect of alcohol on Shh transport in ligand-producing cells, applicant first compared the distribution of Shh among the cellular proteins fractionated by density gradient ultracentrifugation to the distribution of lipid rafts (Caveolin-1), and Golgi and ER compartment markers.

In FIG. 9, alcohol disturbs Shh co-localization with Caveolin-1 in lipid rafts and causes Shh to accumulate in Golgi organelles. To facilitate analysis of the distribution of Shh, cellular proteins were fractionally isolated by density gradient ultracentrifugation; its distribution was compared to the distribution of a lipid raft protein (Caveolin-1), ER and Golgi compartment markers. In untreated cells, Western blot analysis indicated that Shh protein was located in density gradient fractions 7 to 17 (FIG. 4A, top panel); fractions 7 to 11 contained lipid raft fractions as indicated by the presence of Caveolin-1 (FIG. 4A, middle panel); fractions 12 to 17 correspond to the Golgi/ER compartments as indicated by the presence of the Golgi marker (FIG. 4A, third panel). In HSC 8B cells exposed to 0.8% w/v alcohol for 30 minutes, the distribution of Shh shifted out of the Caveolin-1/lipid raft and smooth ER fractions, and was restricted to density gradient fractions 12 to 17, which correspond to the Golgi-associated protein and rough ER fractions (FIG. 4B, top panel).

In protein extracts isolated from cells that were not exposed to alcohol, Western blot analyses indicated that Shh was broadly distributed in the density gradient, from fraction 7 through 17 (FIG. 9A, top panel); in these extracts, lipid raft associated proteins were distributed in fractions 7 through 11, as indicated by the presence of Caveolin-1 (FIG. 9A, second panel). Golgi-associated proteins were present in fractions 12 through 17 (FIG. 9A, third panel); and endoplasmic reticulum-associated proteins were located in fractions of 6 through 9 and 16 and 17 (FIG. 9A, bottom panel), as demonstrated by the presence of specific Golgi/ER markers. In protein extracts isolated from HSC 8B cells that were exposed to 0.6% alcohol (w/v, 109 mM) for 30 minutes, the density gradient distribution of Shh was restricted to fractions 12 through 17 (FIG. 9B, top panel); these fractions contain proteins associated with the Golgi and rough ER (FIG. 9B, third panel), indicating that alcohol exposure shifts Shh distribution away from the lipid raft-containing (FIG. 9B, middle panel) and smooth ER compartment fractions (FIG. 9B, bottom panel).

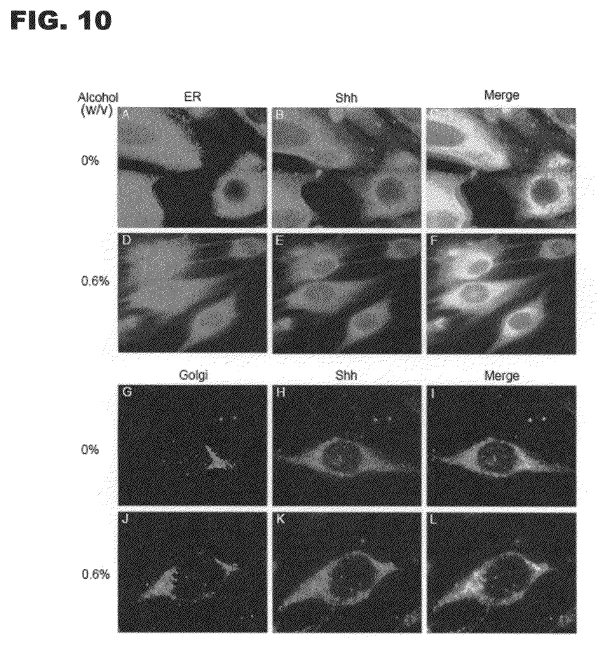

In FIG. 10, alcohol is shown to disrupt Shh entry into ER compartments and causes Shh to accumulate in Golgi organelles. Cells were prepared for immunohischemistry analysis by co-staining with anti-Shh antibody and anti-ER or anti-Golgi marker antibodies or both. In untreated cells, the ER marker (PID) (A, green) and Shh (B, red) co-localized in the cytoplasm as punctate particles (C, yellow). However, in cells exposed to 0.6% (w/v) alcohol for one hour, although the expression levels of the ER marker (D, green) and Shh (E, red) were unchanged, the punctate, polarized distribution pattern of Shh was not detected; instead applicant observed a defused homogeneous distribution of Shh (E and F). G-I: no alcohol exposure; J-L: 0.6% (W/V) alcohol exposure for 1 hour. H and K: anti-Shh antibody staining (red); G and J: anti-Golgi marker antibody staining (green) and I, L: merged corresponding images (yellow). Alcohol treatment did not affect the Shh expression level or its distribution in the Golgi compartment.

To further elucidate the defects that alcohol exposure causes in Shh secretion, applicant used confocal microscopy to examine cells stained with anti-Shh antibodies and antibodies against either Golgi or ER compartment markers. In untreated cells, the ER marker (identified using an anti-PID antibody) (FIG. 10A, green) and Shh (FIG. 10B, red) co-localized in the cytoplasm in punctate particles (FIG. 10C, yellow); however, when HSC 8B cells were exposed to 0.6% (w/v) alcohol for one hour, although the ER marker (FIG. 10D, green) and Shh (FIG. 10E, red) levels were not significantly effected, rather than a punctate, polarized distribution pattern, applicant observed a diffuse, homogeneous distribution pattern for Shh (FIG. 10F). Under the same experimental conditions, applicant determined whether Shh (FIGS. 10E and 10K, red) and the Golgi marker (FIGS. 10G and 10J, green) co-localized by overlaying the corresponding images (FIGS. 10I and 10L, yellow) and found that alcohol treatment did not significantly alter Shh distribution in the Golgi compartment. Hence, the observation that Shh accumulates in the Golgi with no specific entry into the smooth ER indicates that the primary defect in Shh transport caused by alcohol exposure occurs in trafficking between the Golgi and the smooth ER compartments. Previously, applicant demonstrated that alcohol exposure inhibits the post-translational modification of Shh by cholesterol, decreases the amount of the mature Shh ligand that is associated with the cell membrane, and leads to a spectrum of defects in our zebra fish model that phenocopies the defects observed in patients having Fetal Alcohol Syndrome (Li, 2007). In this study, applicant has investigated the mechanism underlying alcohol inhibition of the Hedgehog signal transduction in further detail. Applicant has determined that alcohol exposure leads to defective intracellular transport of the Shh ligand by inhibiting the ability of Shh to form a complex with Caveolin-1, preventing its translocation to the plasma membrane in lipid raft domains.

### Alcohol Exposure Disrupts Shh Secretion

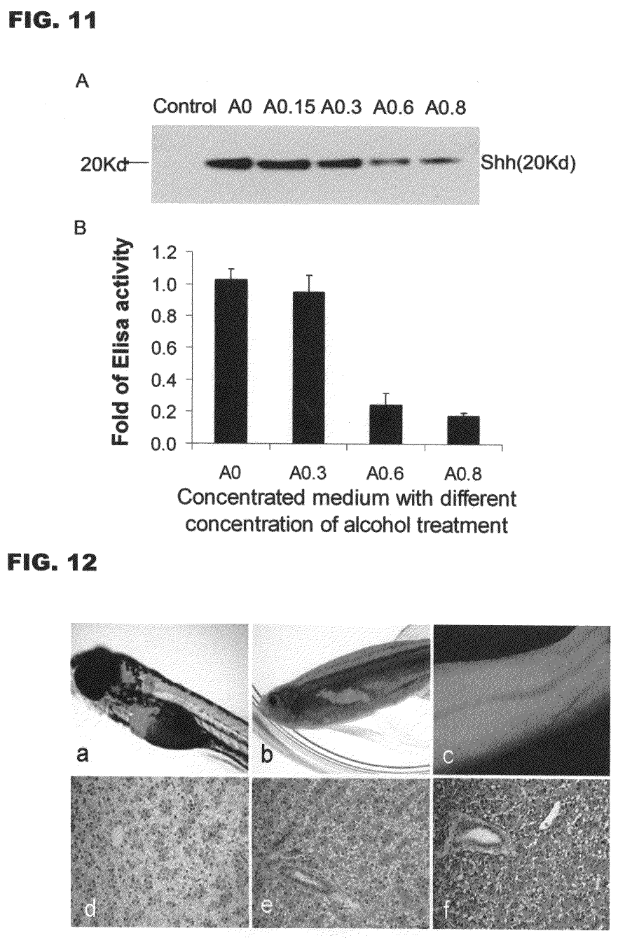

Alcohol exposure inhibits Shh in the Golgi from entering the smooth ER and, therefore, prevents the formation of a Shh/Caveolin-1 protein complex in lipid raft domains, which leads to defective transport of the Shh ligand to the plasma membrane and results in Shh accumulation in the Golgi compartment. Applicant deduced that defective intracellular transport of Shh can lead to decreased secretion of the Shh ligand into the extracellular matrix. To confirm this hypothesis, applicant focused on the effect of alcohol exposure on Shh accumulation in the medium of a cultured Shh producing cell line, HSC 8B. Applicant analyzed proteins collected from the HSC 8B culture medium for Shh ligand content using two independent methods: Western blot analysis and Elisa assay. When the density of HSC 8B cells in culture dishes reached 75% confluence, applicant replaced the culture medium with fresh medium containing serum replacement and various concentrations of alcohol (0, 0.15, 0.3, 0.6 and 0.8% w/v corresponding to 0, 25, 55, 109 and 136 mM). The cultures were incubated for an additional 3 hours, and culture medium was then harvested and concentrated for protein isolation. As shown in FIG. 11A, Western blot analysis of proteins that accumulated in the culture medium indicated that alcohol inhibits Shh secretion in a dose-dependent manner; Elisa assays indicated similar results. In detail, exposure of HSC 8B cells to 0.3%, 0.6% and 0.8% (55, 109 and 136 mM) alcohol concentrations corresponded to 1.2, 4.2 and 5.5 fold decreases in Shh secretion (FIG. 11B). Our results delineate a molecular mechanism for alcohol inhibition of the hedgehog signal transduction pathway in which alcohol inhibits cholesterol-modification of Shh, which hinders Shh binding to Caveolin-1, prevents its entry into the smooth ER, and disrupts its subsequent transport to the plasma membrane in lipid raft domains, resulting in decreased Shh secretion into the extracellular matrix.

### Cholesterol for Stem Cell Nutrients

Cholesterol and its derivatives are nutrients for maintaining physiological function of Hedgehog (Hh) dependent stem cells in embryo and adult tissue. Hedgehog ligands and receptor are expressed in the liver. Hh-responsive cells exist in early embryonic stages, but rarely in adult normal liver.

### Transgenic Zebra Fish with Labelled Mature Liver Cells

Transgenic zebra fish (LFABP-GFP) express GFP in mature hepatocytes and cholangiocytes. A transgenic zebra fish, LFABP-GFP, is the model used for searching for hepatic stem cells. In this transgenic line, all mature hepatocytes and cholangiocytes are labeled with GFP protein via expression driven by the liver fatty acid binding protein (LFABP) promoter.

In FIG. 11, alcohol exposure disrupts Shh secretion into the extracellular matrix. Medium from the cultured HSC 8B cell line was collected and concentrated for use in Western blot analyses (A) and Elisa assays (B). Exposure to various alcohol concentrations (0.15%, 0.3%, 0.6% and 0.8% V/V corresponding to 25, 81, 109, and 136 mM) for 3 hours inhibited Shh secretion in a dose-dependent manner. * indicates a p<0.05.

In FIG. 12, merged fluorescent images show GFP is specifically expressed in liver. a). 2.5 Days embryo, b, c) 8 month adult fish. Immuno-staining on adult liver sections confirms that GFP resembles the endogenous LFABP expression located in mature hepatocytes and cholangiocytes. d) wildtype liver stained with GFP antibody. e,f) transgenic liver stained with GFP (e) and LFABP (f) antibodies.

GFP expression in these cells is initiated on embryonic day 2 (FIG. 12) and is maintained throughout the entire life span. As shown in FIGS. 12B and 12C, the GFP labeled liver can be clearly observed in the living adult zebra fish (6.5 months) under fluorescence microscopy. High levels of GFP expression can be seen in whole liver surgically removed from the fish, blood vessels excepted. Using anti-GFP antibody, immunohistological analyses of liver sections revealed that mature hepatocytes and cholangiocytes in these fish express GFP; moreover, this GFP expression pattern recapitulates the endogenous expression pattern of LFABP that is expressed in hepatocytes and cholangiocytes, but not in nonparenchyma (FIGS. 12E and 12F). Fluorescence activated cell sorting (FACS) was used to separate liver cells into two populations. The GFP positive population contained mature hepatocytes and cholangiocytes. The putative hepatic stem cells were located in the GFP negative fraction. Since GFP is controlled by a specific gene (LFABP) promoter, the GFP expression is restricted in differentiated hepatocytes and cholangiocytes.

This unique feature provides an ideal means for monitoring whether GFP negative cell differentiate into GFP positive cells in an ex vivo transplantation model. When GFP negative/Ptc positive cells are transplanted into a wild type fish, the emergence of GFP positive cells in the recipient liver indicates that some of the donor cells have differentiated, becoming mature hepatocytes or cholangiocytes or both. Therefore, it also allows dynamic monitoring of the differentiation process of transplanted cells in the recipient fish.

### Transgenic Zebra Fish

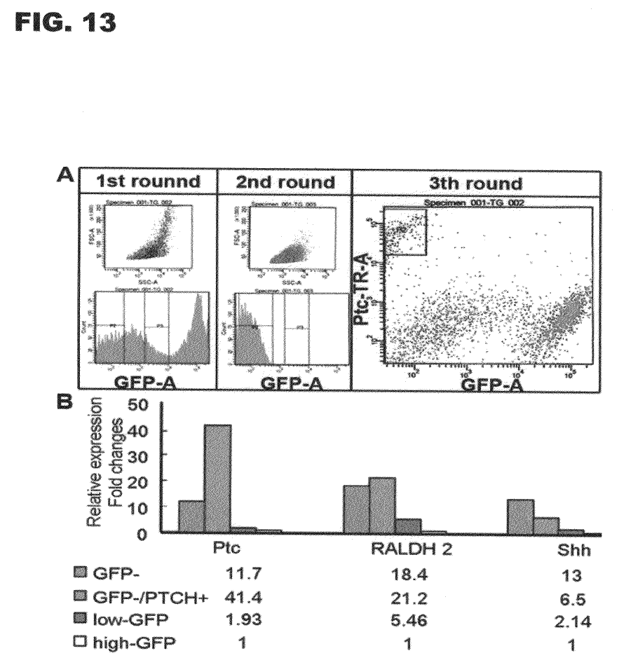

In the transgenic zebra fish, nonparenchymal Ptc positive cells (GFP−/Ptc+), which comprise 0.05% of the adult liver cell population, are morphologically different than mature hepatocytes. Ptc positive (Ptc+) cells were isolated from the GFP negative (GFP−) fraction of the transgenic liver cell population. First, the LFABP-GFP liver was perfused, and then FACS were used to sort out GFP− cells twice from GFP+ cells. Second, GFP− fraction was immunostained with anti-Ptc antibody, followed by secondary antibody incubation which is conjugated with Rhodamine florescence (FIG. 13A). Therefore, the GFP−/Ptc+ cells (that have no green fluorescent/high red fluorescent) were isolated by another round of FACS.

In FIG. 13, cell cytometry is used to isolate GFP−/Ptc+ cell from LFABP-GFP liver (A). Gene expression analysis by real-time quantitative RT-PCR shows that Ptc-antibody sorted GFP− cells are enriched with transcripts of Ptc and Aldh2, but not Shh (B).

In summary, GFP−/Ptc+ cells comprise about 0.05% of the whole liver cell population. Compared to mature hepatocytes, which are about 12-18 μm in diameter, GFP−/Ptc+ cells are small, having diameters of about 4-6 μm. Real time quantitative RT-PCR analysis (FIG. 13B) confirmed that these cells express high levels of Ptc mRNA, 27-fold higher than mature hepatocytes. Another stem cell marker gene, Aldh2, is enriched in GFP−/Ptc+ cells (20-fold higher than expression levels in GFP+ mature hepatocytes and cholangiocytes).

### GFP−/PTC+Cells Differentiate into GPF+ Cells In Vitro

In FGF/HGF hepatic inducing medium, cultured GFP−/Ptc+ cells express GFP and differentiate into hepatocyte-like morphology. Different culture times have been shown in a) 1 hour; b) 5 days; and c,d) 14 days.

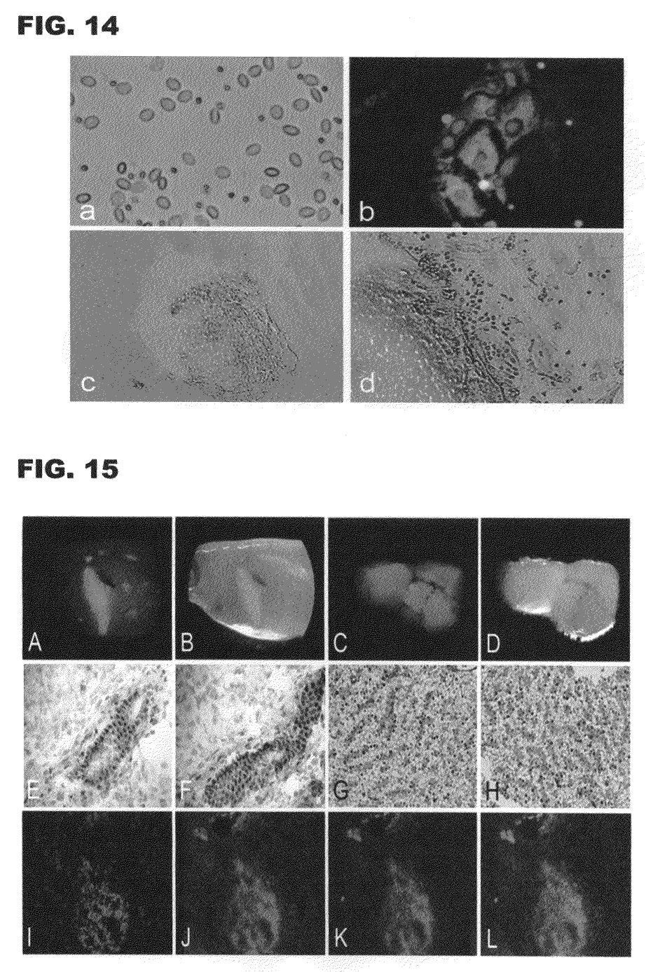

The GFP−/Ptc+ cells (FIG. 14A) are difficult to culture in typical culture medium; no cell divisions occur in the first two weeks in culture, and gradually the cells die. After trying several different culture conditions, these cells were found to prosper in collagen IV and laminin coated culture dishes incubated at 28.5° C. A hepatocyte-inducing medium was formulated that contains 100 ng/ml FGF1, 20 ng/ml FGF4 and 50 ng/ml HGF. In this medium, GFP−/Ptc+ cells start to express GFP, indicating that the cells have differentiated into hepatocytes or cholangiocytes or both.

After culturing these cells for 5 days, cells started to express GFP and transformed like hepatocyte (FIG. 14B). After 14 days in culture, colonies were formed in which cells in the center of the colony express GFP (FIG. 14C); a band of GFP negative cells, 7-9 cells wide, surround the GFP positive cells. These GFP negative cells at the edge of the colonies become GFP positive after an additional 2-3 days, but new GFP negative cells emerged (or migrated) to form a new GFP negative band of cells at the edge of the colonies (FIGS. 14C and 14D).

### GFP−/PTC+Cells Proliferate in Pre-Injured Liver and Differentiate into Biliary Ductural Epithelial Cell and Hepatocyte

In FIG. 15, GFP−/Ptc+ cells integrate into bile ductular epithelial cells and hepatocytes and begin to express GFP in pre-injured liver after transplanted into wild type recipient fishes. Fluorescent images of the recipient fish (A-B) and liver (C, D) one month after transplantation. E-H). GFP antibody immunostaining on recipient liver sections one week (E, H) and one month after transplantation. I-L) Immuno-fluorescence of GFP protein with GFP antibody on one-month-recipient liver sections. I. DAPI nuclear staining, J. endogenous GFP, K. GFP antibody staining; L. merged of 1-K.

To determine whether these small-sized GFP−/Ptc+ cells are multipotent hepatic stem cells, the fate of these cells was investigated by transplanting them into wild type zebra fish and medaka. The day before transplanting the cells, the recipient zebra fish were injected with Tunicamycin, a protein translation inhibitor, to induce extensive liver injury and hepatocyte death. One hundred donor cells (GFP−/Ptc+) were injected intraperitoneally into a recipient wild fish. One week after transplantation, GFP expression was observed in recipient fish when examined under fluorescent microscope. Frozen sections of the recipient liver showed that GFP positive cells had repopulated the liver. Furthermore, GFP monoclonal antibody staining revealed that the donor cells were undergoing very rapid proliferation and differentiation into biliary ductular epithelial cells and some into hepatocytes (FIGS. 15E and 15F). One month after transplantation, the recovered liver contained many GFP positive (FIG. 15L) hepatocytes (10%) that were descendents of the transplanted GFP−/Ptc+donor cells (FIGS. 15G and 15H). This clearly illustrates, as was seen in cell culture, that when transplanted into adult zebra fish with previously injured livers, Ptc positive nonparenchymal cells can differentiate into hepatocyte- and cholangio-like cells. These results provide a solid basis for testing the overall hypothesis of this project: that Hh signaling may regulate self-renewal, expansion and differentiation of stem cells in the adult liver.

### Alcohol Disrupts Shh Ligand with Lipid Raft Association, Inhibits Shh Protein Secretion and Transportation

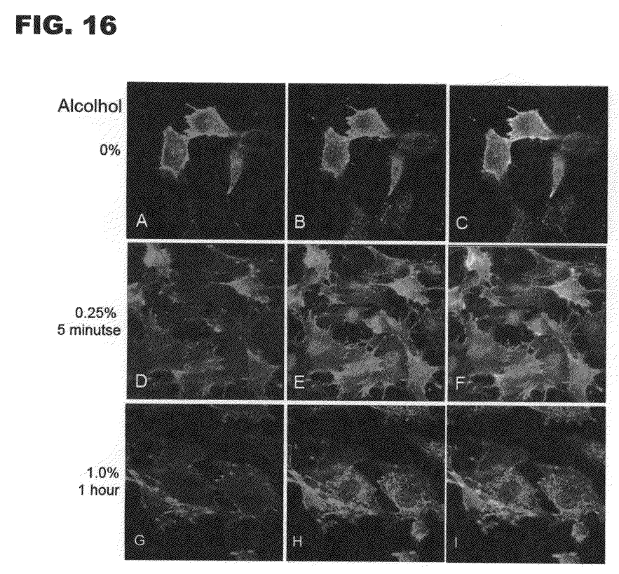

Alcohol disrupts Shh protein (red) and lipid raft (green) co-localization (yellow) and results in Shh transportation and secretion defects as seen in FIG. 16. A-C are the controls without alcohol treatment; D-E treated with 0.25% (V/V) alcohol for 5 minutes; G-I treated with 1.0% alcohol for 1 hour. Lipid raft is labeled by green fluorescent in confocal images of A, D and G; Shh protein is shown by red fluorescent in B, E and H. Merged images are C, F and I in which the yellow signals indicates of the co-localization of Shh and lipid rafts.

In order dissect the detail mechanism of alcohol induced hedgehog signaling defect, the Shh protein transportation in cell was monitored (FIG. 16). A hepatic stellate cell line, HSC8B, from adult rat liver, was chosen as the model to study the dynamic changes of Shh trafficking and co-localization of lipid rafts. Lipid raft was labeled with Vybrant Lipid Raft Labeling Kits (Molecular Probe, Catalog number V34403). This labeling system provides convenient, reliable and extremely bright fluorescent labeling of lipid rafts in live cells.

Lipid rafts are detergent-insoluble, sphingolipid- and cholesterol-rich membrane microdomains that form lateral assemblies in the plasma membrane. It uses the nature affinity of a bacterial toxic protein, cholera toxin subunit B (CT-B), that secreted from Vibrio cholerae bacterium and can specifically binds a constitutional lipid of lipid raft. The Vybrant Lipid Raft Labeling Kits provide the key reagents for fluorescently labeling lipid rafts in vivo with bright and extremely photostable ALEXA FLUOR dyes. Live cells are first labeled with the green-fluorescent Alexa Fluor 488 (or other color dyes) conjugates of cholera toxin subunit B (CT-B). This CT-B conjugate binds to the pentasaccharide chain of plasma membrane ganglioside GM1, which selectively partitions into lipid rafts. An antibody that specifically recognizes CT-B is then used to crosslink the CT-B labeled lipid rafts into distinct patches on the plasma membrane, which are easily visualized by fluorescence microscopy. When liver stellate cells were treated with alcohol concentration at 0.25% v/v) just for 5 minutes, the co-localization of Shh (red) and lipid raft (green) is disrupted (less yellow comparing to no alcohol treated), when alcohol concentration increased to 1% for one hour, the Shh protein expression level was not affected, but almost all of the Shh protein was accumulated inside of the liver stellate cells, no association with lipid raft or cell membrane.

Therefore the bottom line of the alcohol pathological mechanism has been found that alcohol inhibited cholesterylation of Hedgehog protein, without lipid anchor, the Shh protein could not be associated with lipid raft and failure for secretion; therefore these transportation defects lead to this morphogen gradient abnormality that results in development problems such as fetal alcohol syndrome, as well as liver cirrhosis.

Furthermore, in adult stem cell in many organs, it has been demonstrated that Hedgehog pathway plays a major role for participating tissue regeneration and repairing. These stem cells are found in brain, skin and digestive system. Alcoholism speeds aging process and induces liver damage, even liver cirrhosis. Providing cholesterol and cholesterol derivatives may hold a key to maintain adult stem cell function and prevent alcoholic aging and diseases, such as cirrhosis.

### Summary of Studies

1. Based on applicant's previous work on Ptc-lacZ transgenic mice, applicant noted that the numbers of Ptc positive cells decreased dramatically in mice after embryonic Day 11 during the hepatocyte differentiation. In adult mice, all mature hepatocytes lack Ptc expression, but a very few positive Ptc expressing cells are located near the bile ductular plate. Using sucrose gradient centrifugation, applicant found Ptc positive cells in the nonparenchymal fraction of the liver cell population. More importantly, Ptc positive cells can be induced to proliferate and differentiate under pathological stimulation. Bile ductule ligation leads to a remarkable increase in the proliferation of Ptc positive cells; furthermore, some of these cells are progenitors of the oval cell lineage. These results strongly suggest that a cell membrane protein, the Hh receptor, Ptc, may be useful to identify and isolate quiescent adult hepatic stem cells from adult livers.

2. Given that pathological stimulation induces Ptc positive cell proliferation and differentiation, Ptc positive cells were isolated from adult livers. Using a unique transgenic zebra fish model, Ptc positive cells were purified from the nonparenchymal fraction of the adult liver cell population. These relatively small Ptc positive nonparenchymal cells can be induced to differentiate into hepatocytes and cholangiocytes when transplanted into adult zebra fish having previously injured livers.

3. Liver regeneration is necessary to repair damaged livers, including livers damaged by chronic consumption of alcohol. Accumulating evidence suggests that the pathological mechanism for alcohol-induced defects may involve in impaired Hh signaling. Fetal alcohol administration results in similar abnormalities to those seen in animals having Hh signaling defects or cholesterol metabolic defects. Hh signaling controls the development of the organs or tissues that are also the most vulnerable targets in Fetal Alcohol Syndrome. In zebra fish, applicant has found that alcohol can inhibit Hh signaling by disrupting cholesterol homeostasis, impairing cholesterol-Shh modification and Shh transportation in zebra fish embryo and rat adult liver cell. Supplemental cholesterol rescues cholesterol modification of Shh, restores Hh signaling, and prevents alcohol-induced developmental defects.

### Identification of Biomarkers for Diagnosis Alcohol Related Diseases by Lipid Chemical Analysis and Raman Spectroscope

The consumption of alcohol is on the rise, especially in women. Overall, its effects on the fetal development are more harmful than those attributed to cocaine, heroin or marijuana. In the United States, FAS is the leading cause of mental retardation and congenital defects, surpassing even spina bifida and Down's syndrome. Approximately 50,000 children are born with alcohol-related defects in the U.S annually. Despite prenatal education and general public awareness, one out of five pregnant women is believed to consume alcohol during pregnancy. Moreover, 45% of women who consumed alcohol also reported that they did not learn of their pregnancy until after the fourth week of gestation. As a result, the cost of alcohol-related birth defects is an estimated US$ 9.7 billion annually. Though extensive research work is continuing on FAS, little conclusive evidence is available beyond the documented fact that alcohol is harmful to the developing fetus.

Detecting alcohol use amongst pregnant women is an important step toward preventing alcohol-related birth defects. Since maternal alcohol use is under-reported and identification of alcohol-exposed newborns is often difficult in the absence of severe FAS defects, a biomarker that could detect alcohol use during pregnancy would aid in earlier identification and intervention for pregnant mothers and affected infants. More importantly, early intervention for affected children before the age of six may reduce the incidence of anti-social behavior later in life.

Our observation suggests a novel mechanism for the teratogenic effects of alcohol via alterations in cholesterol metabolism. Even at very low concentrations of alcohol exposure, applicant observed consistent and dramatic decreases in cholesterol ester levels and in the ratio of cholesterol ester-to-total cholesterol in zebra fish embryos. Furthermore, applicant also find impaired signaling in the Hedgehog pathway, which plays a key role in the embryonic development of numerous organs and structures that are vulnerable to prenatal alcohol exposure. This altered signaling appears to be due to defects in the cholesterol modification of Hedgehog ligand. To confirm our findings in a mammalian system, applicant has chronically fed mice an alcohol diet and find similar aberrations in cholesterol homeostasis. These mechanistic studies provide a solid indication that focusing on metabolic profile analysis of free fatty acids and cholesterol will lead to a new set of biomarkers for alcohol exposure.

Based on our previous data, our goal is to identify a biomarker signature that detects maternal and prenatal alcohol exposure by fingerprinting metabolic intermediates, such as cholesterol chemically by clinic test or physically by Raman Spectroscope.

Raman Spectroscope: When light passes through matter, most photons continue in their original direction but a small fraction are scattered in other directions. Light that is scattered due to vibrations is called Raman scattering or the Raman Effect. The difference in energy between the incident photon and the Raman scattered photon is equal to the energy of a vibration of the scattering molecule. A plot of intensity of scattered light versus energy difference is a Raman Spectrum. The measurement of the identity and intensity of Raman Spectrum can specifically identify molecules and their concentration in a complicated system. This is the physiochemical basis of the Raman spectroscope.

### Alcohol Treatment Reduced Cholesterol Ester Content and Cholesterol Transportation

Our previsions data (FIG. 3) demonstrate that alcohol exposure during the late blastula-gastrula stage causes a dose-dependent reduction in membrane-associated Shh. Given that esterification of Shh by cholesterol drives its membrane localization, these results suggest that alcohol exposure reduces cholesterol ester formation. To screen for this possibility, applicant measured cholesterol ester content in whole embryo extracts that were exposed to various doses of alcohol for 3 h during the late blastula-gastrula stage. Applicant next tested whether alcohol exposure impairs general sterol homeostasis during gastrulation by measuring cholesterol levels in whole embryo extracts. In a dose-related fashion, alcohol exposure resulted in a decrease in the total cholesterol content of embryos (FIG. 4). This was mostly due to reductions of cholesterol esters, very minor from the reduction of the free cholesterol.

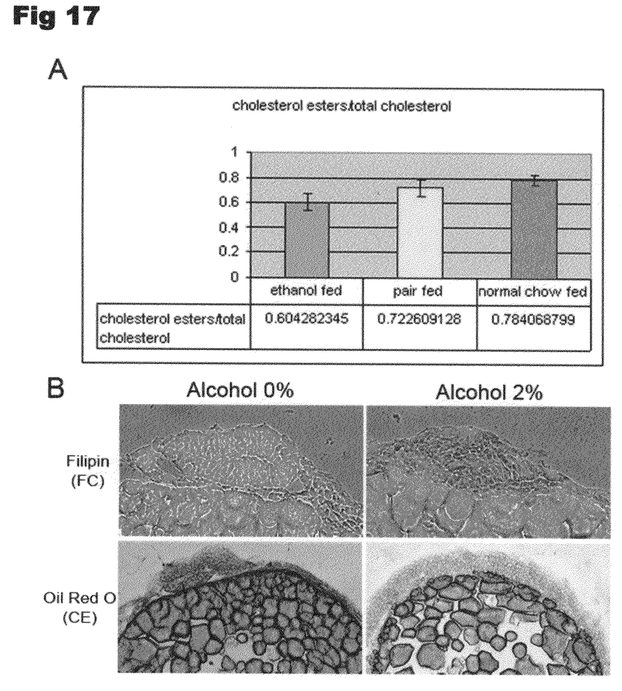

These trends are also seen in the data collected from a chronic alcohol administration in mice model. The ratio of cholesterol ester to total cholesterol in plasma is significantly decreased in the alcohol treated group by comparing both control groups (p<0.001) (FIG. 17A). In FIG. 17, alcohol exposures disrupt free cholesterol/cholesterol ester balance and transport in embryos. A. Chronic alcohol feeding decreases the ratio of cholesterol ester to total cholesterol. B. Filipin staining showed a significant reduction of free cholesterol in embryo body; Oil red O staining identified cholesterol ester remarkable decrease in embryo yolk.

All of the maternal free cholesterol is deposited in the embryonic yolk. Upon esterified reaction, then the cholesterol ester can be transported from the embryonic yolk to the body. In order to investigate whether there is esterification and transportation defect of cholesterol in alcohol treated embryos, applicant further determined the distribution of free cholesterol and cholesterol ester between embryo body and yolk by two chemicals that can differentiate the free cholesterol and cholesterol ester. The fluorescent molecule, Filipin can selectively bind to free cholesterol, but not cholesterol ester; in other hand, the dye of oil red O identifies neutral lipids molecules such as cholesterol ester, but not polarized free cholesterol. First, applicant quantitatively analyzed the free cholesterol by whole mount in situ filipin staining. In situ quantitative density analysis of filipin-free cholesterol staining showed that free cholesterol decreased differentially in alcohol-exposed embryos, showing a large decrease in cholesterol concentration in the embryonic body, but not in the embryonic yolk. Second, oil red O staining revealed a dramatic decrease of cholesterol ester in the embryonic yolk, with little or no change in its concentration in the embryonic body (FIG. 17B). The differential spatial changes in the concentrations of the different forms of cholesterol indicate that inhibition of cholesterol esterification by alcohol may lead to deficient cholesterol transportation from the yolk to the body (tissues), resulting in hypocholesteromia in embryonic tissues, and subsequently defective Shh cholesterol modification.

### Non Invasive Detection of Alcoholic Related Biomarkers by Raman Spectrum

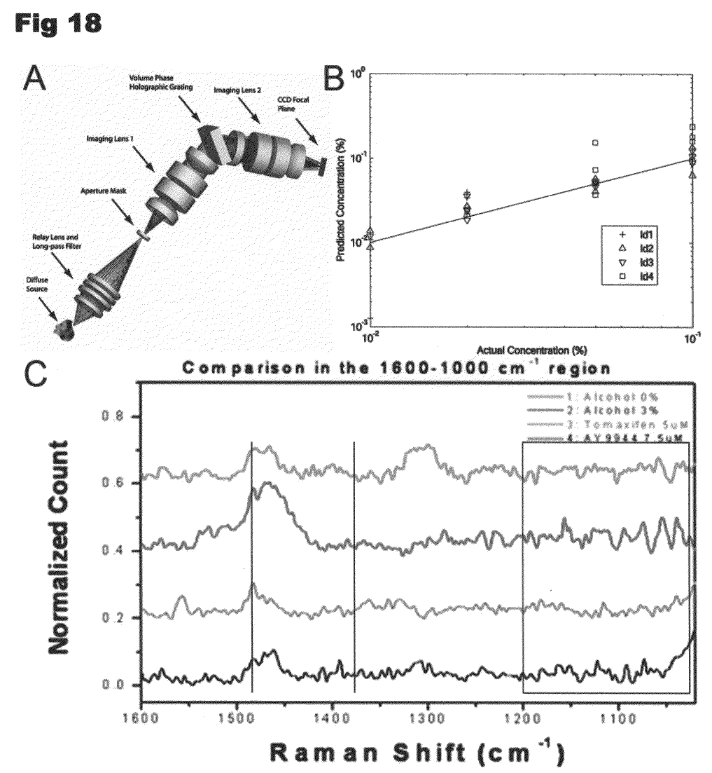

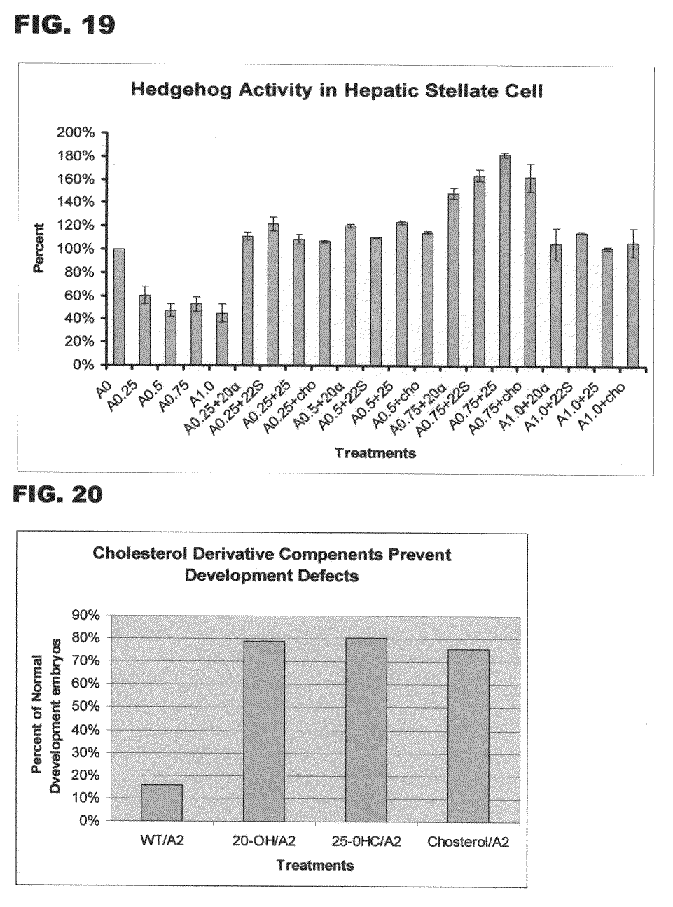

Applicant has developed “Multimodal multiplex multi-wavelength” Raman spectroscopy. This system achieves uniquely high optical throughput and fluorescence rejection for detecting alcohol in tissue as well as tracing alcohol exposure induced cholesterol signature changes. The sensor, a combination of spatially coded detection optics and spectrally coded excitation sources to get the Raman spectrum of alcohol in tissue (FIG. 18A).

FIG. 18 characterizes the alcohol and cholesterol signatures for Raman spectra in vivo in alcohol-exposed embryos: Coded-aperture multi-wavelength Raman spectroscopy (A) and its sensitivity for detecting alcohol in vitro can reach as low as 0.01% (B). Raman signature changes of cholesterol in embryos treated with alcohol (3%), Tomaxifin (5 uM) or AY9944 (7.5 uM) (C).

The plot shows the correlation between measurement principle spectral component amplitudes and concentration of alcohol. The measured results were excellent in accuracy for approximately 10% to 0.01% of tissue alcohol concentrations (FIG. 18B). Applicant has applied the Raman system to test cholesterol in zebra fish embryos. A signature change in cholesterol Raman spectrum has been found that it is related to embryos exposure to alcohol in the Raman spectrum region 1600-1000 cm⁻¹ (FIG. 18C). This alcoholic Raman spectrum is dramatically changed is the increase of the peaks intensity around 1470, and around 1300 cm⁻¹ in which the cholesterol peak is clearly decreased under alcohol influence. Another important difference is the fine Raman features from 1000 to 1200 cm⁻¹, where clear peaks can be detected in alcohol treated embryos. This change of cholesterol Raman spectrum was also unique by comparing other two known cholesterol homeostasis inhibitors: Tomxafin (inhibiting cholesterol esterification) and AY9944 (inhibiting cholesterol biosynthesis). The signatures of these two drugs related Raman spectrum are also having far clinic impact for diagnosis and monitoring cholesterol defects caused by these drugs.

### Cholesterol Derivative Components Rescue Alcohol Inhibited Hedgehog Signaling Activity in Rat Stellate Cells

Cholesterol derivative components rescue alcohol inhibited Hedgehog signaling activity in rat liver stellate cells. Hepatic stellate cells, also known as Ito cells, are found in the perisinusoidal space (a small area between the sinusoids and hepatocytes) of the liver. The stellate cell is the major cell type involved in liver fibrosis, which is the formation of scar tissue in response to liver damage. In normal liver, stellate cells are described as being in a quiescent state. Quiescent stellate cells represent 5-8% of the total number of liver cells. Different environmental factors and disease caused liver injury (such as alcohol exposure) can be activated. The activated stellate cell is responsible for secreting collagen scar tissue (fibrosis), which can lead to cirrhosis.

The Gli-luciferase reporter assay was performed in duplicated experiments of a rat hepatic stellate cell line, 8H. Briefly, 5H cells, were grown in DMEM medium supplemented with 10% fetal bovine serum and penicillin and streptomycin (100 U/ml). At 40-50% confluency, cells were transfected with Gli-BS-Firefly luciferase plasmid (60 ng nl⁻¹) and Renilla luciferase plasmid (60 ng nl⁻¹, pRL-TK, Promega) (concentration ratio 10:1) by using FuGENE 6 Transfection Reagent (Roche Applied Science). Twenty-four hours later, the medium was replaced with medium containing different concentration of alcohol. Two hours later, the medium was added to different drugs (20α-OHC, 22(S)-OHC, 25-OHC and cholesterol). After 16 hrs, the cells were washed three times in ice-cold PBS, and then were assayed by Dual/Luciferase Reporter Assay System (Promega). Activity of the Firefly luciferase reporter was normalized to the activity of a Renilla luciferase internal control for transfection efficiency.

Alcohol treatments (0.25, 0.5, 0.75 and 1.0% v/v) decrease Hedgehog signaling activates as measured by Gli binding site derived luciferase activities in hepatic stellate cells when compared to no alcohol treatment group (A0). After alcohol exposure. 2-3 hours, adding cholesterol as well as other sterol-like components can rescue the Hedgehog pathway function back to normal level like the no-alcohol exposure group (Table II). These tested chemicals and their concentrations region are described in following Table.

TABLE II
CHOLESTEROL AND OTHER CHOLESTEROL SIMILAR
COMPONENTS RESTORE HEDGEHOG SIGNALING ACTIVITY
IN ALCOHOL-TREATED HEPATIC STELLATE CELLS

| | CONCEN- | | | |
|---|---|---|---|---|
| | TRATION | | PREFERRED | |
| COMPONENT | REGION | | CONCENTRATION | |
| Cholesterol | 1-50 | μM | 1-20 | μM |
| 25-hydroxycholesterol | 0.1-100 | μM | 5-15 | μM |
| 22(S)-Hydroxycholesterol | 0.1-100 | μM | 1-5 | μM |
| 20α-Hydroxycholesterol | 0.1-100 | μM | 1-5 | μM |
| Squalene | 0.1-100 | μM | 1-10 | μM |

### Cholesterol Derivative Components Prevent Alcohol Induced Developmental Defects in Zebra Fish Embryo

Cholesterol and cholesterol-like components rescue hedgehog activity in alcohol treated rat hepatic stellate cell in tissue culture system. In order to test the rescuing ability to also function in an animal model, cholesterol and cholesterol-like components were microinjected into zebra fish embryos (concentration regions are listed in Table II above) at 1-2 cell stage with 0.2 nl of cholesterol and cholesterol derivative. Embryos were allowed to develop 4.3 hours, and then treated with alcohol for 6 hours as previously described. At 72 hpf embryos were analyzed.

### Results

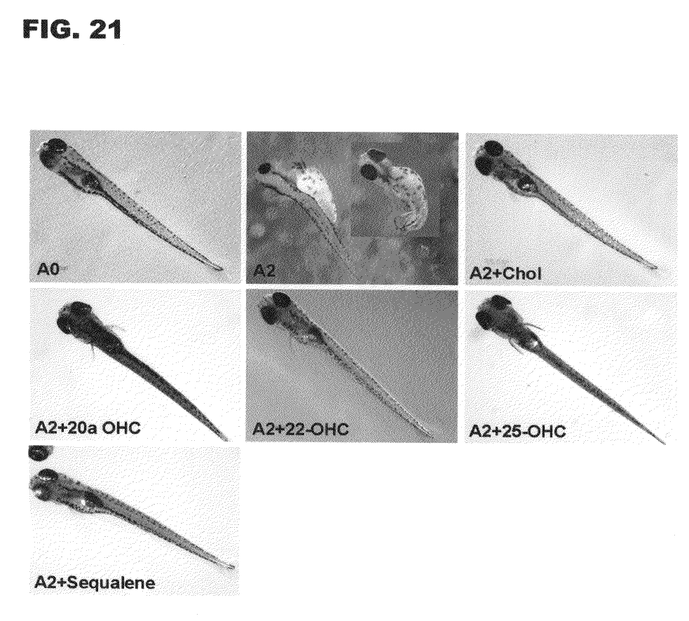

As seen in FIG. 21, cholesterol and cholesterol-like molecules prevent alcohol induced embryonic developmental defects. A0: no alcohol exposure; A2: 2% V/V alcohol exposure. Chol: Cholesterol; 20a OHC: 22α-hydroxycholesterol; 22-OHC: 22-hydroxycholesterol; 25-OHC: 25-hydroxycholesterol and Squalene.

When zebra fish embryos were treated with 2.0% alcohol for 6 hours starting from 4.3 hours after fertilization, it causes development defects such as forebrain truncation, cyclopia and retardation of growth. Only 15% of these alcohol (2%) treated embryo were developed relatively normal on gross morphology. On other hand, these 2% alcohol exposure embryos were supplied with cholesterol and cholesterol-like components, the percent of gross normal developed embryos increased significantly reaching to about 80% (See FIGS. 20 and 21). In FIGS. 20 and 21, cholesterol and cholesterol-like molecules are shown to prevent alcohol induced embryonic developmental defects. AO: no alcohol exposure; A2: 2% V/V alcohol exposure. Chol: Cholesterol; 20a OHC: 22a-hydroxycholesterol; 22-OHC: 22-hydroxycholesterol; 25-OHC; 25-hydrocholesterol and Squalene.

### Cholesterol Treatment for Adult Tissue

### Types of Cholesterol Used

Cholesterol is a sterol (a combination of a steroid and alcohol) and a lipid found in the cell membranes of all body tissues, and transported in the blood plasma of all animals.
1. The chemical name of cholesterol is 10,13-dimethyl-17-(6-methylheptan-2-yl)-2,3,4,7,8,9,11,12,14,15,16,17-dodecahydro-1H-cyclopenta[a]phenanthren-3-ol.
   The chemical formula is C₂₇H₄₆O.
   The chemical structure of cholesterol is illustrated as following.
   The molecular mass is 386.65 g/mol.

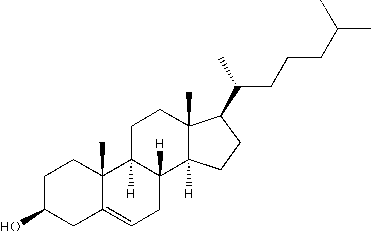

II. 20-hydroxycholesterol

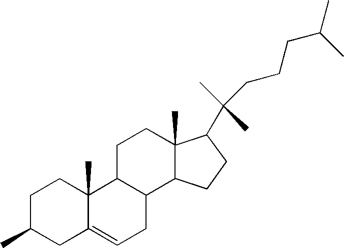

III. NAME: 5-cholestene-3b,22-diol (22-hydroxycholesterol)
   COMMON NAME
   SYMBOL
   FORMULA: C₂₇H₄₆O₂ MOL.WT (average): 402.653

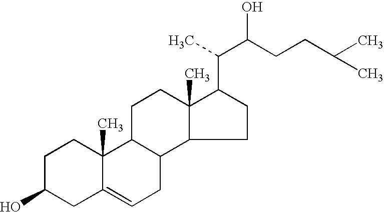

IV. NAME: 5-cholestene-3b,25-diol (25-hydroxycholesterol)
   COMMON NAME
   SYMBOL
   FORMULA: C₂₇H₄₆O₂ MOL.WT (average): 402.653
V. Squalene.
   NAME: 2,6,10,15,19,23-Hexamethyl-2,6,20,24,28,22-Tetracosahexaene
   COMMON NAME: Squalene/Spinacene/Supraene
   SYMBOL
   FORMULA: C₃₀H₅₀ MOL.WT. (average): 410.718

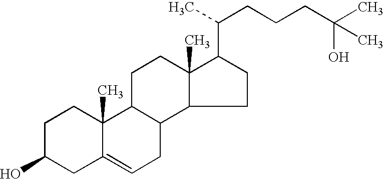

Sigma Company manufactures these cholesterols, most of them being used for research purposes. Four of these molecules have been tested to date which have a similarity of cholesterol structure.

### Stem Cell Nutrition

A combination of regular cholesterol and one of the other four (above) would be safe for over-the-counter supplement for supporting good t-cell nutrition. To date, cholesterol and other four forms tested have the ability to maintain function of stem cells that are dependent on hedgehog signaling in fish embryos and cultured liver cell line. These functions are rescuing development defects induced by environmental factors such as alcohol and statins, and function through improving cell survival ability, proliferation and regeneration ability: Stem Cell Nutrition.

### Nutrient Supplement and Pharmaceutical

Overall, cholesterol is a natural molecule in our body and intake cholesterol from food on a daily basis. Cholesterol, even cholesterol-like substances, can be marketed as a nutrient. All of these components also have great potential to be developed as new drug.

These cholesterol compounds may be in conflict with cholesterol lowering products such as LIPITOR which shows a lot of side effects. The major use of LIPITOR is lowering cholesterol when it is over 220 mg/dl. Lowering cholesterol to a very low level will damage stem cells and related tissue regeneration and aging. Possible side effects of an increased cholesterol regimen might be high cholesterol level in blood and tissue. The other cholesterol-like molecules may have a chance to produce too much stem cells in the body and therefore it may has high chance to produce tumor.

### Liver Treatment

Cholesterol treatment for liver damage provides more hedgehog activity for hepatic stellate cells. Oral pill cholesterol form should be the most common and convenient way. Muscle or vein injection can be used for special cases.

### Treatment of Other Medical Conditions

Here is a list of some medical conditions treatable by OTC or prescription cholesterol:
Cancer patient after chemotherapy and radiotherapy;
Statin lowering cholesterol too low;
Bone marrow transplantation;
Stem cell transplantation therapy
Aging patient has nutrient problem,
Losing memory
Depress and stress
Pregnancy
Pregnancy drinking alcohol

### Bone Marrow Treatment

Cholesterol treatment is shown herein to prevent overall whole embryo development defects, but there is not direct evidence for bone marrow defect benefits. Exogenous supplement cholesterol is the general approach, taken orally, skin delivered, and by muscle or vein injection. Cholesterol was delivered by injection as reported herein. Data on bone marrow effectiveness is yet available. It had been proven how Hedgehog signaling effects bone marrow stem cell by maintaining or rescuing hedgehog signaling activity. Some medical conditions for which the OTC or the prescription cholesterol treatments could help are as follows:
Leukemia patient with bone marrow transplantation;
Other cancer patients after chemotherapy or radiotherapy;
Children with blood stem cell problem.

### Neurons in the Brain

In zebra fish, lab investigation shows how cholesterol prevents overall whole embryo development defects, specifically for forebrain and neural tube and neural tube defect. Exogenous supplement cholesterol is the general approach, taken orally, skin delivered, and muscle or vein injection. Our data for forebrain and eye defects and their rescuing approach are presented in the published paper.

We show dose region of individual cholesterols function on hedgehog signaling on fish and rat liver cell line. These observations show that the efficacy order is 25-OHC, 22(S)-OHC, cholesterol, 20α-OHC and followed with squalene using regular cholesterol. Some medical conditions for which the OTC or the prescription cholesterol treatments could help are brain injury, most of chronic brain aging and disease.

### Other Organs

Other body organs which could be helped using the cholesterol and cholesterol-like therapy treatments of this invention are:
Skin
Pancreas
G.I. Tract

While a number of exemplary aspects and embodiments have been discussed above, those of skill in the art will recognize certain modifications, permeations and additions and subcombinations thereof. It is therefore intended that the following appended claims and claims hereinafter introduced are interpreted to include all such modifications, permeations, additions and subcombinations that are within their true spirit and scope.

### REFERENCES

1. Mann R K, Beachy P A. Novel lipid modifications of secreted protein signals. Annu Rev Biochem 2004; 73:891-923.
2. Hammerschmidt M, Brook A, McMahon A P. The world according to hedgehog. Trends Genet. 1997; 13:14-21.
3. Nybakken K, Perrimon N. Heparan sulfate proteoglycan modulation of developmental signaling in Drosophila. Biochim Biophys Acta 2002; 1573:280-91.
4. Porter J A, Young K E, Beachy P A. Cholesterol modification of hedgehog signaling proteins in animal development. Science 1996; 274:255-9.
5. Feng J, White B, Tyurina O V, Guner B, Larson T, Lee H Y, Karlstrom R O, Kohtz J D. Synergistic and antagonistic roles of the Sonic hedgehog N- and C-terminal lipids. Development 2004; 131:4357-70.
6. Stone D M, Hynes M, Armanini M, Swanson T A, Gu Q, Johnson R L, Scott M P, Pennica D, Goddard A, Phillips H, Noll M, Hooper J E, de Sauvage F, Rosenthal A. The tumour-suppressor gene patched encodes a candidate receptor for Sonic hedgehog. Nature 1996; 384:129-34.
7. Ding Q, Fukami S, Meng X, Nishizaki Y, Zhang X, Sasaki H, Dlugosz A, Nakafuku M, Hui C. Mouse suppressor of fused is a negative regulator of sonic hedgehog signaling and alters the subcellular distribution of Gli1. Curr Biol 1999; 9:1119-22.
8. Concordet J P, Lewis K E, Moore J W, Goodrich L V, Johnson R L, Scott M P, Ingham P W. Spatial regulation of a zebrafish patched homologue reflects the roles of sonic hedgehog and protein kinase A in neural tube and somite patterning. Development 1996; 122:2835-46.
9. Dai P, Akimaru H, Tanaka Y, Maekawa T, Nakafuku M, Ishii S. Sonic Hedgehog-induced activation of the Gli1 promoter is mediated by GLI3. J Biol Chem 1999; 274:8143-52.
10. Barth K A, Wilson S W. Expression of zebrafish nk2.2 is influenced by sonic hedgehog/vertebrate hedgehog-1 and demarcates a zone of neuronal differentiation in the embryonic forebrain. Development 1995; 121:1755-68.
11. Echelard Y, Epstein D J, St-Jacques B, Shen L, Mohler J, McMahon J A, McMahon A P. Sonic hedgehog, a member of a family of putative signaling molecules, is implicated in the regulation of CNS polarity. Cell 1993; 75:1417-30.
12. Roelink H, Augsburger A, Heemskerk J, Korzh V, Norlin S, Ruiz i Altaba A, Tanabe Y, Placzek M, Edlund T, Jessell T M, et al. Floor plate and motor neuron induction by vhh-1, a vertebrate homolog of hedgehog expressed by the notochord. Cell 1994; 76:761-75.
13. Riddle R D, Johnson R L, Laufer E, Tabin C. Sonic hedgehog mediates the polarizing activity of the ZPA. Cell 1993; 75:1401-16.
14. Johnson R L, Laufer E, Riddle R D, Tabin C. Ectopic expression of Sonic hedgehog alters dorsal-ventral patterning of somites. Cell 1994; 79:1165-73.
15. Wallis D, Muenke M. Mutations in holoprosencephaly. Hum Mutat 2000; 16:99-108.
16. Ronen G M, Andrews W L. Holoprosencephaly as a possible embryonic alcohol effect. Am Med Genet. 1991; 40:151-4.
17. Siebert J R, Astley S J, Clarren S K. Holoprosencephaly in a fetal macaque (Macaca nemestrina) following weekly exposure to ethanol. Teratology 1991; 44:29-36.
18. Coulter C L, Leech R W, Schaefer G B, Scheithauer B W, Brumback R A. Midline cerebral dysgenesis, dysfunction of the hypothalamic-pituitary axis, and fetal alcohol effects. Arch Neurol 1993; 50:771-5.
19. Croen L A, Shaw G M, Lammer E J. Risk factors for cytogenetically normal holoprosencephaly in California: a population-based case-control study. Am J Med Genet 2000; 90:320-5.
20. Belloni E, Muenke M, Roessler E, Traverso G, Siegel-Bartelt J, Frumkin A, Mitchell H F, Donis-Keller H, Helms C, Hing A V, Heng H H, Koop B, Martindale D, Rommens J M, Tsui L C, Scherer S W. Identification of Sonic hedgehog as a candidate gene responsible for holoprosencephaly. Nat Genet 1996; 14:353-6.
21. Ericson J, Morton S, Kawakami A, Roelink H, Jessell T M. Two critical periods of Sonic Hedgehog signaling required for the specification of motor neuron identity. Cell 1996; 87:661-73.
22. Roebuck T M, Simmons R W, Richardson C, Mattson S N, Riley E P. Neuromuscular responses to disturbance of balance in children with prenatal exposure to alcohol. Alcohol Clin Exp Res 1998; 22:1992-7.
23. Loser H, Bierstedt T, Blum A. [Fetal alcohol syndrome in adulthood. A long-term study]. Dtsch Med Wochenschr 1999; 124:412-8.
24. Kyllerman M, Aronson M, Sabel K G, Karlberg E, Sandin B, Olegard R. Children of alcoholic mothers. Growth and motor performance compared to matched controls. Acta Paediatr Scand 1985; 74:20-6.
25. Connor P D, Sampson P D, Streissguth A P, Bookstein F L, Barr H M. Effects of prenatal alcohol exposure on fine motor coordination and balance: A study of two adult samples. Neuropsychologia 2006; 44:744-51.
26. Chudley A E, Conry J, Cook J L, Loock C, Rosales T, LeBlanc N. Fetal alcohol spectrum disorder: Canadian guidelines for diagnosis. Cmaj 2005; 172:S1-S21.
27. Wada N, Javidan Y, Nelson S, Carney T J, Kelsh R N, Schilling T F. Hedgehog signaling is required for cranial neural crest morphogenesis and chondrogenesis at the midline in the zebrafish skull. Development 2005; 132:3977-88.
28. Mann R K, Beachy P A. Cholesterol modification of proteins. Biochim Biophys Acta 2000; 1529:188-202.
29. Zeng X, Goetz J A, Suber L M, Scott W J, Jr., Schreiner C M, Robbins D J. A freely diffusible form of Sonic hedgehog mediates long-range signalling. Nature 2001; 411:716-20.
30. Cooper M K, Porter J A, Young K E, Beachy P A. Teratogen-mediated inhibition of target tissue response to Shh signaling. Science 1998; 280:1603-7.
31. Leiber B. [Alcohol—currently the most significant agent causing malformations. Severe multiple abnormalities in children of alcoholics are increasing at an alarming rate. A warning from DOFONOS]. Med Monatsschr Pharm 1978; 1:25-6.
32. Jones K L, Smith D W. Recognition of the fetal alcohol syndrome in early infancy. Lancet 1973; 2:999-1001.
33. Olney J W, Tenkova T, Dikranian K, Muglia L J, Jermakowicz W J, D'Sa C, Roth K A. Ethanol-induced caspase-3 activation in the in vivo developing mouse brain. Neurobiol Dis 2002; 9:205-19.
34. Charness M E, Safran R M, Perides G. Ethanol inhibits neural cell-cell adhesion. J Biol Chem 1994; 269:9304-9.
35. Kotch L E, Chen S Y, Sulik K K. Ethanol-induced teratogenesis: free radical damage as a possible mechanism. Teratology 1995; 52:128-36.
36. Henderson G I, Baskin G S, Horbach J, Porter P, Schenker S. Arrest of epidermal growth factor-dependent growth in fetal hepatocytes after ethanol exposure. J Clin Invest 1989; 84:1287-94.
37. Deltour L, Ang H L, Duester G. Ethanol inhibition of retinoic acid synthesis as a potential mechanism for fetal alcohol syndrome. Faseb J 1996; 10:1050-7.
38. Blader P, Strahle U. Ethanol impairs migration of the prechordal plate in the zebrafish embryo. Dev Biol 1998; 201:185-201.
39. Yelin R, Schyr R B, Kot H, Zins S, Frumkin A, Pillemer G, Fainsod A. Ethanol exposure affects gene expression in the embryonic organizer and reduces retinoic acid levels. Dev Biol 2005; 279:193-204.
40. Solnica-Krezel L. Conserved patterns of cell movements during vertebrate gastrulation. Curr Biol 2005; 15:R213-28.
41. Tanaka Y, Okada Y, Hirokawa N. FGF-induced vesicular release of Sonic hedgehog and retinoic acid in leftward nodal flow is critical for left-right determination. Nature 2005; 435:172-7.
42. Barresi M J, Stickney H L, Devoto S H. The zebrafish slow-muscle-omitted gene product is required for Hedgehog signal transduction and the development of slow muscle identity. Development 2000; 127:2189-99.
43. Kelley R L, Roessler E, Hennekam R C, Feldman G L, Kosaki K, Jones M C, Palumbos J C, Muenke M. Holoprosencephaly in RSH/Smith-Lemli-Opitz syndrome: does abnormal cholesterol metabolism affect the function of Sonic Hedgehog? Am J Med Genet 1996; 66:478-84.
44. Dehart D B, Lanoue L, Tint G S, Sulik K K. Pathogenesis of malformations in a rodent model for Smith-Lemli-Opitz syndrome. Am J Med Genet 1997; 68:328-37.
45. Cooper M K, Wassif C A, Krakowiak P A, Taipale J, Gong R, Kelley R I, Porter F D, Beachy P A. A defective response to Hedgehog signaling in disorders of cholesterol biosynthesis. Nat Genet 2003; 33:508-13.
46. Polo M P, de Bravo M G, de Alaniz M J. Effect of ethanol on cell growth and cholesterol metabolism in cultured Hep G2 cells. Biochem Cell Biol 2003; 81:379-86.
47. Axelson M, Mork B, Sjovall J. Ethanol has an acute effect on bile acid biosynthesis in man. FEBS Lett 1991; 281:155-9.
48. Buchmann M S, Kvittingen E A, Nazer H, Gunasekaran T, Clayton P T, Sjovall J, Bjorkhem I. Lack of 3 beta-hydroxy-delta 5-C27-steroid dehydrogenase/isomerase in fibroblasts from a child with urinary excretion of 3 beta-hydroxy-delta 5-bile acids. A new inborn error of metabolism. J Clin Invest 1990; 86:2034-7.
49. Gigliozzi A, Romeo R, Fraioli F, Cantafora A, Delle Monache M, Cardilli A, Attili A F, Scafato E, Carli L, Alvaro D. Effect of S-adenosyl-L-methionine and dilinoleoylphosphatidylcholine on liver lipid composition and ethanol hepatotoxicity in isolated perfused rat liver. Dig Dis Sci 1998; 43:2211-22.
50. Carrasco M P, Segovia J L, Marco C. Incorporation of exogenous precursors into neutral lipids and phospholipids in rat hepatocytes: effect of ethanol in vitro. Biochem Pharmacol 1998; 56:1639-44.
51. Wilbanks A M, Fralish G B, Kirby M L, Barak L S, Li Y X, Caron M G. Beta-arrestin 2 regulates zebrafish development through the hedgehog signaling pathway. Science 2004; 306:2264-7.
52. Sicklick J K, Li Y X, Choi S S, Qi Y, Chen W, Bustamante M, Huang J, Zdanowicz M, Camp T, Torbenson M S, Rojkind M, Diehl A M. Role for hedgehog signaling in hepatic stellate cell activation and viability. Lab Invest 2005; 85:1368-80.
53. Sasaki H, Hui C, Nakafuku M, Kondoh H. A binding site for Gli proteins is essential for HNF-3beta floor plate enhancer activity in transgenics and can respond to Shh in vitro. Development 1997; 124:1313-22.
54. Crow M T, Stockdale F E. Myosin expression and specialization among the earliest muscle fibers of the developing avian limb. Dev Biol 1986; 113:238-54.
55. Bilotta J, Barnett J A, Hancock L, Saszik S. Ethanol exposure alters zebrafish development: a novel model of fetal alcohol syndrome. Neurotoxicol Teratol 2004; 26:737-43.
56. Bilotta J, Saszik S, Givin C M, Hardesty H R, Sutherland S E. Effects of embryonic exposure to ethanol on zebrafish visual function. Neurotoxicol Teratol 2002; 24:759-66.
57. Arenzana F J, Carvan M J, 3rd, Aijon J, Sanchez-Gonzalez R, Arevalo R, Porteros A. Teratogenic effects of ethanol exposure on zebrafish visual system development. Neurotoxicol Teratol 2006; 28:342-8.
58. Carvan M J, 3rd, Loucks E, Weber D N, Williams F E. Ethanol effects on the developing zebrafish: neurobehavior and skeletal morphogenesis. Neurotoxicol Teratol 2004; 26:757-68.
59. Reimers M J, Flockton A R, Tanguay R L. Ethanol- and acetaldehyde-mediated developmental toxicity in zebrafish. Neurotoxicol Teratol 2004; 26:769-81.
60. Dasmahapatra A K, Doucet H L, Bhattacharyya C, Carvan M J, 3rd. Developmental expression of alcohol dehydrogenase (ADH3) in zebrafish (Danio rerio). Biochem Biophys Res Commun 2001; 286:1082-6.
61. Reimers M J, Hahn M E, Tanguay R L. Two zebrafish alcohol dehydrogenases share common ancestry with mammalian class I, II, IV, and V alcohol dehydrogenase genes but have distinct functional characteristics. J Biol Chem 2004; 279:38303-12.
62. Sulik K K, Johnston M C, Webb M A. Fetal alcohol syndrome: embryogenesis in a mouse model. Science 1981; 214:936-8.
63. Ikonomidou C, Bittigau P, Ishimaru M J, Wozniak D F, Koch C, Genz K, Price M T, Stefovska V, Horster F, Tenkova T, Dikranian K, Olney J W. Ethanol-induced apoptotic neurodegeneration and fetal alcohol syndrome. Science 2000; 287:1056-60.
64. Von Knappen B, Thater, H., Loeser, H. Alkoholschaeden bei Kindern. Ratgeber zur Alkoholembryopathie. Lambertus Verlag Freiburg, Germany, 1996.
65. Ahlgren S C, Thakur V, Bronner-Fraser M. Sonic hedgehog rescues cranial neural crest from cell death induced by ethanol exposure. Proc Natl Acad Sci USA 2002; 99:10476-81.
66. Corcoran R B, Scott M P. Oxysterols stimulate Sonic hedgehog signal transduction and proliferation of medulloblastoma cells. Proc Natl Acad Sci USA 2006; 103:8408-13.
67. Pepinsky R B, Zeng C, Wen D, Rayhorn P, Baker D P, Williams K P, Bixler S A, Ambrose C M, Garber E A, Miatkowski K, Taylor F R, Wang E A, Galdes A. Identification of a palmitic acid-modified form of human Sonic hedgehog. J Biol Chem 1998; 273:14037-45.
68. Kohtz J D, Lee H Y, Gaiano N, Segal J, Ng E, Larson T, Baker D P, Garber E A, Williams K P, Fishell G. N-terminal fatty-acylation of sonic hedgehog enhances the induction of rodent ventral forebrain neurons. Development 2001; 128:2351-63.
69. Lee J D, Treisman J E. Sightless has homology to transmembrane acyltransferases and is required to generate active Hedgehog protein. Curr Biol 2001; 11:1147-52.
70. Lewis P M, Dunn M P, McMahon J A, Logan M, Martin J F, St-Jacques B, McMahon A P. Cholesterol modification of sonic hedgehog is required for long-range signaling activity and effective modulation of signaling by Ptc1. Cell 2001; 105:599-612.
71. Chen M H, Li Y J, Kawakami T, Xu S M, Chuang P T. Palmitoylation is required for the production of a soluble multimeric Hedgehog protein complex and long-range signaling in vertebrates. Genes Dev 2004; 18:641-59.

1. A method of detecting the presence of alcohol or cholesterol lowering components and damage associated therewith in embryonic and adult tissue and organs comprising determining the level of defectiveness which has occurred to Shh protein in the tissue and organs due to the alcoholic or cholesterol lowering components.

2. A method of detecting alcohol-damaged embryonic and/or adult Shh protein and hedgehog pathway activity comprising:
detecting and analysis of defects of cholesterol-hedgehog protein modification, Shh/Caveolin-1 binding ability, Shh intracellular trafficking, plasma membrane and lipid raft association, secretion, intercellular transportation, gradient establish, and hedgehog pathway signal transduction, cholesterol profile signature and Raman spectrum thereof.

3. A method for reducing a condition associated with fetal alcohol syndrome in a subject exposed to alcohol in utero, the method comprising:
administering the subject a cholesterol or a cholesterol derivative in an amount sufficient to reduce the condition associated with fetal alcohol syndrome.

4. A method for screening and identifying one or more agents which are protective or therapeutic for fetal alcohol syndrome and adult stem cell aging related defects, comprising:
administering the agent to a zebra fish embryo model or rat hepatic stellate cell lines before, after or concurrently with the transient alcohol exposure of the embryo; and
detecting a serial of molecular and cellular defects in the alcohol treated embryo compared to a control.
